(19) United States
(12) Patent Application Publication
(10) Pub. No.: US 2022/0281802 A1
(43) Pub. Date: Sep. 8, 2022

(54) COMPOUNDS, PHARMACEUTICAL COMPOSITIONS, AND METHODS OF THEIR USE IN THE INHIBITION OF INTERACTION BETWEEN IL18 AND IL18R

(71) Applicant: Beth Israel Deaconess Medical Center, Inc., Boston, MA (US)

(72) Inventors: Leena PRADHAN-NABZDYK, Rochester, MN (US); Lijun SUN, Harvard, MA (US); Philip LOGERFO, Cambridge, MA (US); Finith JERNIGAN, Brookline, MA (US); Gabriel BIRANNE, Lynn, MA (US); Frank W. LOGERFO, Cambridge, MA (US)

(21) Appl. No.: 17/631,972

(22) PCT Filed: Jul. 31, 2020
(86) PCT No.: PCT/US2020/044636

§ 371 Date: Feb. 1, 2022

Related U.S. Application Data
(60) Provisional application No. 62/881,679, filed on Aug. 1, 2019.

(51) Int. Cl. C07C 211/45; C07D 249/18; C07C 279/18; C07D 401/04

(52) U.S. Cl. CPC C07C 211/45; C07D 249/18; C07C 279/18; C07D 401/04; C07C 2601/16

(57) ABSTRACT

Compounds, pharmaceutical compositions, and methods are disclosed for inhibiting interaction between IL18 and IL18R.

### STATEMENT AS TO FEDERALLY FUNDED RESEARCH

The invention was made with Government support under Grant No. HL133821 awarded by the National Institutes of Health. The government has certain rights in the invention.

### FIELD OF THE INVENTION

The invention relates to compounds, pharmaceutical compositions, and methods of their use in the inhibition of interaction between interleukin-18 (IL18) and the interleukin-18 receptor (IL18R).

### BACKGROUND

Interleukin-18 (IL18) is a pleiotropic pro-inflammatory cytokine belonging to the IL-1 superfamily. IL18 plays an important role in host innate and adaptive immune defense against pathogenic infections. Despite the significant role of IL18 in host immune responses against infection, aberrant hIL18 bioactivity also has been associated with inflammatory and autoimmune disorders, allergies, and neurological disorders. In fact, it has been shown that increased levels of mature hIL18 have a direct correlation with the severity of pathological autoimmune disorders such as Multiple Sclerosis (MS), Rheumatoid Arthritis (RA), and lupus. A current strategy for treating these human diseases is to target proteins involved in the initiation event(s) of inflammation or upstream events of the innate immune response. These upstream effector proteins include but are not limited to Cyclooxygenase-2 (Cox-2) and caspase-1, which respond to Non-Steroidal Anti-Inflammatory Drugs (NSAID) and specific caspase inhibitors, respectively. However, these treatments suffer from side effects, such as colitis.

IL18 was originally referred to as IFN-γ Inducing Factor (IGIF) for its ability to stimulate the production of IFN-γ. IL18 stimulates IFN-γ production from T-helper lymphocytes (Th1) and macrophages, and enhances the cytotoxicity of natural killer (NK) cells. The IL18 stimulated IFN-γ production is synergistically amplified with other Th1-related cytokines, IL-2, IL-15, IL-12, and IL-23. IL18 is synthesized as a 23 kDa inactive precursor, which is subsequently cleaved into an 18 kDa active form by a member of the inflammasome (Interleukin-1β Converting Enzyme, ICE (Caspase-1) and then secreted, resulting in the initiation of IL18 signaling cascade. IL18 signals through its two membrane bound receptors, IL18Rα and IL18Rβ, forming a ternary complex necessary for productive intracellular signaling.

IL18 activity is modulated in vivo by its naturally occurring antagonist, Interleukin-18 Binding Protein (IL18BP), a soluble protein including a single immunoglobulin (Ig) domain. The human IL18BP (hIL18BP) has an exceptionally high affinity for hIL18 of 400 pM and has been shown to be up-regulated in various cell lines in response to elevated IFN-γ levels, suggesting that it serves as a negative feedback inhibitor of hIL18 mediated immune responses. hIL18BP has also been shown to be effective at treating inflammatory skin diseases and lipopolysaccharide (LPS)-induced liver injury, but has also met with complications often causing immunogenic reaction themselves.

There is a need for IL18 inhibitors for, e.g., the treatment of inflammatory or autoimmune disorders and cancer.

### SUMMARY OF THE INVENTION

In one aspect, the invention features a compound according to formula (I):

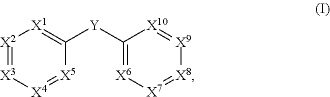

or a pharmaceutically acceptable salt thereof,

### Where:

each of X¹, X², X³, X⁴, X⁵, X⁶, X⁷, X⁸, X⁹, and X¹⁰ is independently CR¹ or N;

Y is O, C(R²)₂, or S(O)ₜ;

each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R² is independently hydrogen or optionally substituted C₁-C₆ alkyl; or both R² combine to form ═O, ═S, or ═NR⁵;

R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R⁴ is independently optionally substituted C₁-C₆ alkyl, optionally substituted C₆-C₁₀ aryl, optionally substituted heteroaryl, or optionally substituted heterocyclyl;

R⁵ is hydrogen, hydroxyl, —S(O)₂R⁴, optionally substituted C₁-C₆ alkyl;

each t is independently 0, 1, or 2; and

In particular embodiments, the compound of formula (I) is not a compound selected from the group consisting of:

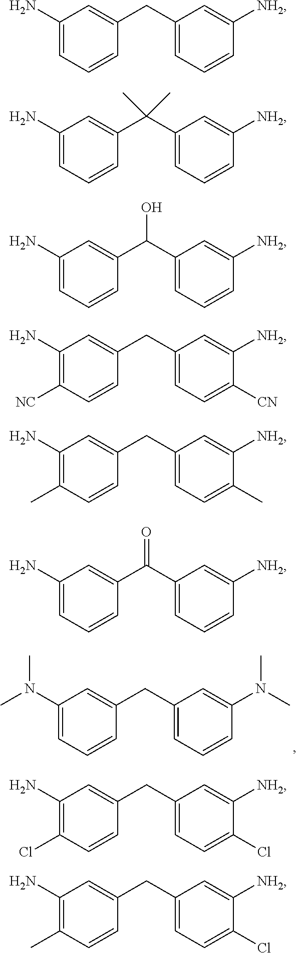

and pharmaceutically acceptable salts thereof.

In some embodiments, Y is C(R²)₂, and R² is at least one hydrogen. In other embodiments R² combine to form ═O, or each is independently optionally substituted C₁-C₆ alkyl.

In certain embodiments, each of X² and X⁹ is independently CR¹. In other embodiments, at least two R¹ groups are —N(R³)₂.

In further embodiments, the compound has the structure of formula (Ia):

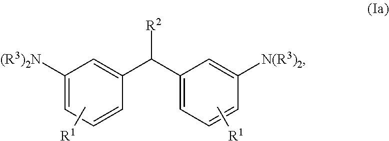

or a pharmaceutically acceptable salt thereof,
where:

each R¹ is independently halogen, hydroxyl, cyano, nitro, —N(R³)₂, —S(O)ₜR⁴, —OR³, optionally substituted C₁-C₆ alkyl, optionally substituted C₆-C₁₀aryl, optionally substituted C₃-C₈cycloalkyl, or optionally substituted heteroaryl.

In some embodiments, the compound has the structure of formula (Ib):

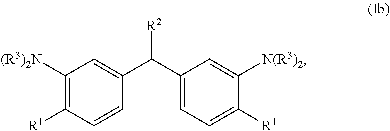

or a pharmaceutically acceptable salt thereof.

In certain embodiments, R² is hydrogen, hydroxyl, or optionally substituted C₁-C₆ alkyl.

In an aspect, the invention features a pharmaceutical composition including a compound of formula (I):

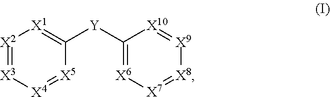

or a pharmaceutically acceptable salt thereof,
where:

each of X¹, X², X³, X⁴, X⁵, X⁶, X⁷, X⁸, X⁹, and X¹⁰ is independently CR¹ or N;

Y is O, C(R²)₂, or S(O)ₜ;

each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R² is independently hydrogen, hydroxyl, or optionally substituted C₁-C₆alkyl; or both R² combine to form ═O, ═S, or ═NR⁵;

R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R⁴ is independently optionally substituted C₁-C₆ alkyl, optionally substituted C₆-C₁₀ aryl, optionally substituted heteroaryl, or optionally substituted heterocyclyl;

R⁵ is hydrogen, hydroxyl, —S(O)₂R⁴, optionally substituted or C₁-C₆ alkyl; and each t is independently 0, 1, or 2.

In certain embodiments, Y is C(R²)₂, and R² is at least one hydrogen. In other embodiments, R² combine to form ═O, or each is independently optionally substituted C₁-C₆ alkyl.

In certain embodiments, each of X² and X⁹ is independently CR¹. In other embodiments, at least two R¹ groups are —N(R³)₂.

In further embodiments, the compound of a pharmaceutical composition has the structure of formula (Ia):

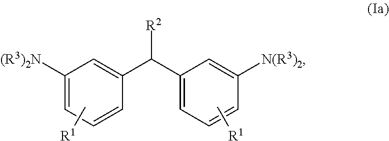

or a pharmaceutically acceptable salt thereof,
where:

each R¹ is independently halogen, hydroxyl, cyano, nitro, —N(R³)₂, —S(O)ₜR⁴, —OR³, optionally substituted C₁-C₆ alkyl, optionally substituted C₆-C₁₀aryl, optionally substituted C₃-C₈cycloalkyl, or optionally substituted heteroaryl.

In some embodiments, the compound of a pharmaceutical composition has the structure of formula (Ib):

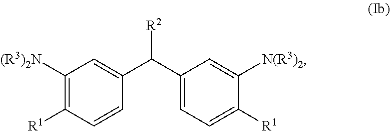

or a pharmaceutically acceptable salt thereof.

In certain embodiments, R² is hydrogen, hydroxyl, or optionally substituted C₁-C₆ alkyl.

In some embodiments, the compound of formula (I) in a pharmaceutical composition is selected from the group consisting of:

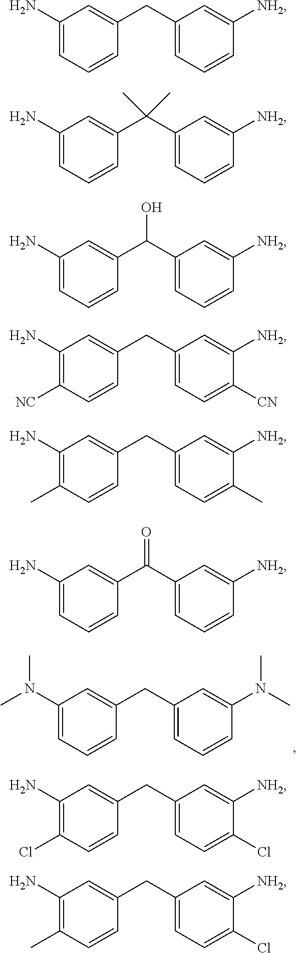

and pharmaceutically acceptable salts thereof.

In one aspect, the invention features a compound of formula (II):

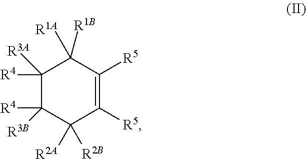

or a pharmaceutically acceptable salt thereof,
where:

(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or

(ii) each of R^{1A} and R^{2A} is independently hydrogen, halogen, hydroxyl, cyano, nitro, —S(O)ₜR⁶, or —OR⁷; R^{1B} and R^{3A}, together with the atoms to which they are attached, combine to form a double bond; and R^{2B} and R^{3B}, together with the atoms to which they are attached, combine to form a double bond;

both R⁴, together with the atoms to which they are attached, combine to form an optionally substituted heterocyclic ring;

both R⁵, together with the atoms to which they are attached, combine to form an optionally substituted heterocyclic ring;

each R⁶ is independently hydrogen, halogen, cyano, or optionally substituted C₁-C₆ alkyl; each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl; and

each t is independently 0, 1, or 2.

In some embodiments, the compound of formula (II) is not a compound selected from the group consisting of:

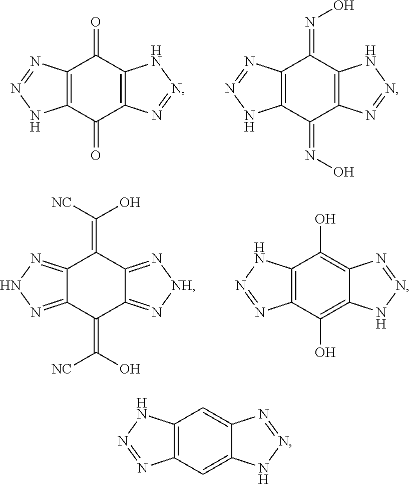 [SMILES: O=C1c2nn[nH]c2C(=O)c2nn[nH]c21 ; O/N=C1/c2nn[nH]c2/C(=N/O)c2nn[nH]c21 ; N#CC(O)=c1c2n[nH]nc2c(=C(O)C#N)c2n[nH]nc12 ; Oc1c2nn[nH]c2c(O)c2nn[nH]c12 ; c1c2nn[nH]c2cc2nn[nH]c12]

and pharmaceutically acceptable salts thereof.

In certain embodiments, both R⁴ together with the atoms to which they are attached combine to form an optionally substituted heterocyclic ring including an endocyclic nitrogen atom. In other embodiments, both R⁴ together with the atoms to which they are attached combine to form an optionally substituted 5-membered heterocyclic ring.

In some embodiments, both R⁵ together with the atoms to which they are attached combine to form an optionally substituted heterocyclic ring containing an endocyclic nitrogen atom. In further embodiments, both R⁵ together with the atoms to which they are attached combine to form an optionally substituted 5-membered heterocyclic ring.

In further embodiments, the compound has the structure of formula (IIa):

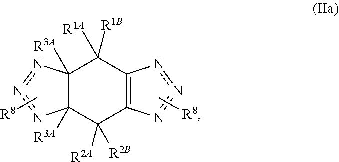

or a pharmaceutically acceptable salt thereof, and each R⁸ is independently H or optionally substituted C₁-C₆ alkyl.

In some embodiments, R^{1A} and R^{1B} combine to form ═O. In some embodiments, R^{2A} and R^{2B} combine to form ═O. In particular embodiments, each of R^{1A} and R^{2A} is independently hydrogen, halogen, or hydroxyl.

In an aspect, the invention features a pharmaceutical composition including a compound of formula (II):

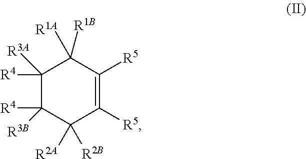

or a pharmaceutically acceptable salt thereof,
where:

(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or

(ii) each of R^{1A} and R^{2A} is independently hydrogen, halogen, hydroxyl, cyano, nitro, —S(O)R⁶, or —OR⁷; R^{1B} and R^{3A}, together with the atoms to which they are attached, combine to form a double bond; and R^{2B} and R^{3B}, together with the atoms to which they are attached, combine to form a double bond; both R⁴, together with the atoms to which they are attached, combine to form an optionally substituted heterocyclic ring;

both R⁵, together with the atoms to which they are attached, combine to form an optionally substituted heterocyclic ring;

each R⁶ is independently hydrogen, halogen, cyano, or optionally substituted C₁-C₆ alkyl; each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl; and each t is independently 0, 1, or 2.

In certain embodiment, both R⁴ together with the atoms to which they are attached combine to form an optionally substituted heterocyclic ring including an endocyclic nitrogen atom. In further embodiments, both R⁴ together with the atoms to which they are attached combine to form an optionally substituted 5-membered heterocyclic ring.

In particular embodiments, both R⁵ together with the atoms to which they are attached combine to form an optionally substituted heterocyclic ring containing an endocyclic nitrogen atom. In other embodiments, both R⁵ together with the atoms to which they are attached combine to form an optionally substituted 5-membered heterocyclic ring.

In further embodiments, the compound of a pharmaceutical composition has the structure of formula (IIa):

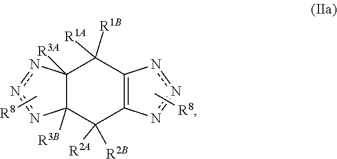

or a pharmaceutically acceptable salt thereof, and each R⁸ is independently H or optionally substituted C₁-C₆ alkyl.

In some embodiments, R^{1A} and R^{1B} combine to form ═O. In some embodiments, R^{2A} and R^{2B} combine to form ═O. In particular embodiments, each of R^{1A} and R^{2A} is independently hydrogen, halogen, or hydroxyl.

In some embodiments, the compound of formula (II) in a pharmaceutical composition is selected from the group consisting of:

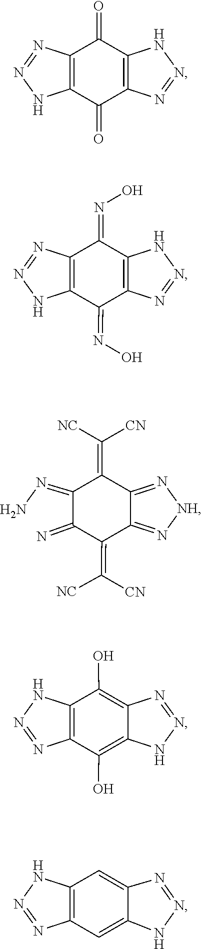

and pharmaceutically acceptable salts thereof.

In one aspect, the invention features a compound of formula (III):

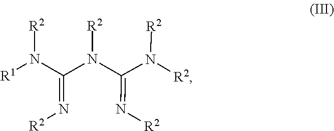

or a pharmaceutically acceptable salt thereof,
where:

R¹ is an optionally substituted C₆-C₁₀ aryl or optionally substituted heteroaryl;

each R² is independently hydrogen or an optionally substituted C₁-C₆ alkyl; and

In some embodiments, the compound of formula (III) is not a compound selected from the group consisting of:

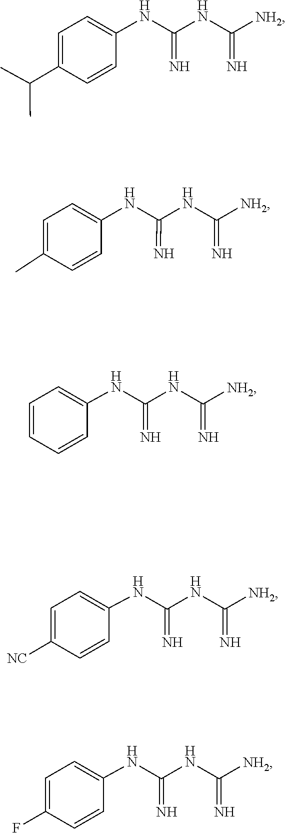 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]

and pharmaceutically acceptable salts thereof.

In some embodiments, R¹ is optionally substituted C₆-C₁₀ aryl. In certain embodiments, each R² is hydrogen.

In further embodiments, the compound has the structure of formula (IIIa):

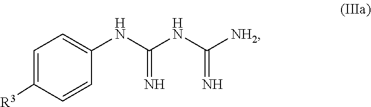

or a pharmaceutically acceptable salt thereof,
where:

R³ is optionally substituted C₁-C₆ alkyl, halogen, —OR⁴, —S(O)R⁵, or —N(R⁴)₂;

each R⁴ is independently hydrogen or optionally substituted C₁-C₆ alkyl;

R⁵ is optionally substituted C₁-C₆ alkyl, optionally substituted C₆-C₁₀ aryl, or optionally substituted heteroaryl; and

each t is independently 0, 1, or 2.

In particular embodiments, R³ is optionally substituted C₁-C₆ alkyl or halogen.

In an aspect, the invention features a pharmaceutical composition including a compound of formula (III):

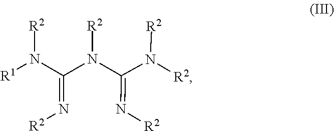

or a pharmaceutically acceptable salt thereof,
where:

R¹ is an optionally substituted C₆-C₁₀ aryl or optionally substituted heteroaryl; and

each R² is independently hydrogen or an optionally substituted C₁-C₆ alkyl.

In some embodiments, R¹ is optionally substituted C₆-C₁₀ aryl. In certain embodiment, each R² is hydrogen.

In further embodiments, the compound of a pharmaceutical composition has the structure of formula (IIIa):

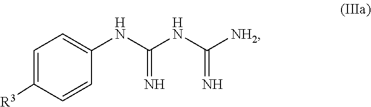

or a pharmaceutically acceptable salt thereof,
where:

R³ is optionally substituted C₁-C₆ alkyl, halogen, —OR⁴, —S(O)R⁵, or —N(R⁴)₂;

each R⁴ is independently hydrogen or optionally substituted C₁-C₆ alkyl;

R⁵ is optionally substituted C₁-C₆ alkyl, optionally substituted C₆-C₁₀ aryl, or optionally substituted heteroaryl; and

each t is independently 0, 1, or 2.

In some embodiments, R³ is optionally substituted C₁-C₆ alkyl, hydrogen, halogen, or cyano.

In particular embodiments, the compound of formula (III) of a pharmaceutical composition is selected from the group consisting of:

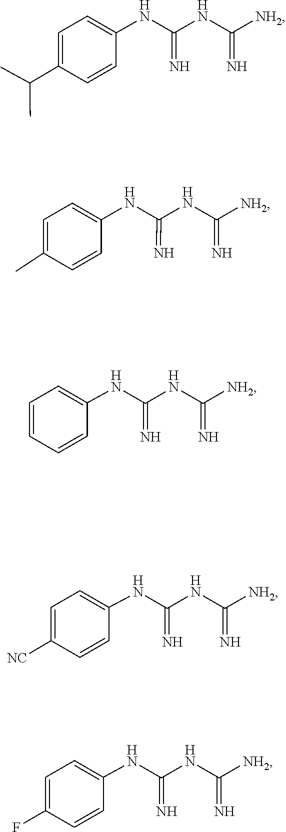 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]

and pharmaceutically acceptable salts thereof.

In one aspect, the disclosure features a method of inhibiting interaction between IL18 and IL18R in a medium including IL18 and a cell expressing IL18R by contacting the cell in the medium with a compound disclosed herein. After the contacting step the interaction between IL18 and IL18R is inhibited.

In some embodiments, the cell is in a subject. In some embodiments, the medium is a tissue or bodily fluid of a subject.

In another aspect, the disclosure features a method of treating an inflammatory or autoimmune disorder in a subject in need thereof by administering a therapeutically effective amount of the compound disclosed herein, or a pharmaceutically acceptable salt thereof, or the pharmaceutical composition disclosed herein to the subject.

In some embodiments, the inflammatory or autoimmune disorder is selected from the group consisting of rheumatoid arthritis, osteoarthritis, lupus, renal disease, psoriasis, inflammatory bowel disease, vascular graft failure, heart disease, vascular disease, type 1 diabetes, type 2 diabetes, metabolic syndrome, diabetic wound healing, and kidney disease. In certain embodiments, the inflammatory or autoimmune disorder is a vascular graft failure. In particular embodiments, the vascular graft failure is a peripheral vascular graft failure. In further embodiments, the peripheral vascular graft failure is a vein graft failure or prosthetic graft failure. In yet further embodiments, the vascular graft failure is a coronary artery graft failure. In still further embodiments, the coronary graft failure is an is artery graft failure, vein graft failure, prosthetic graft failure. In other embodiments, the vascular graft failure is restenosis after stent graft. In yet other embodiments, the vascular graft failure is restenosis after balloon angioplasty. In still other embodiments, the inflammatory or autoimmune disorder is a heart disease. In some embodiments, the heart disease is cardiomyopathy, congestive heart failure, or ischemic coronary heart disease. In certain embodiments, the inflammatory or autoimmune disorder is a vascular disease. In particular embodiments, the vascular disease is intimal hyperplasia, atherosclerosis, coronary artery disease, restenosis, primary hypertension, secondary hypertension, peripheral vascular disease, or critical limb-threatening ischemia. In further embodiments, the inflammatory or autoimmune disorder is type 1 diabetes, type 2 diabetes, metabolic syndrome, or diabetic wound healing. In yet further embodiments, the inflammatory or autoimmune disorder is a kidney disease. In still further embodiments, the kidney disease is a chronic kidney disease, end-stage renal disease, or kidney failure.

In some embodiments, the inflammatory or autoimmune disorder is sepsis.

In some embodiments, the inflammatory or autoimmune disorder is a cytokine storm.

In some embodiments, the inflammatory or autoimmune disorder is endothelialitis. In some embodiments, the subject is suffering from acute respiratory distress syndrome (ARDS) (e.g., ARDS associated with an infection (e.g., a viral infection, such as a beta-coronavirus infection or an influenza infection)). In some embodiments, the inflammatory or autoimmune disorder is associated with an infection (e.g., a viral infection, bacterian infection, protozoan infection, or fungal infection). In some embodiments, the inflammatory or autoimmune disorder is associated with a viremia. In some embodiments, the inflammatory or autoimmune disorder is associated with a bacteremia. In some embodiments, the inflammatory or autoimmune disorder is associated with a protozoan infection. In some embodiments, the inflammatory or autoimmune disorder is associated with a fungal infection.

In some embodiments, the infection is a beta-coronavirus infection. In some embodiments, the infection is SARS-CoV-2, SARS-CoV, or MERS-CoV. In some embodiments, the infection is SARS-CoV-2.

In some embodiments, the subject has been diagnosed with SARS-CoV-2 infection.

In some embodiments, the subject has Covid-19 disease.

In some embodiments, the subject is an asymptomatic carrier of SARS-CoV-2.

In some embodiments, the subject has one or more symptoms of COVID-19 selected from the group consisting of fever, sore throat, runny nose, sneezing, nasal congestion, snoring, coughing, dry cough, shortness of breath, difficulty breathing, persistent pain or pressure in the chest, dyspnea, pneumonia, acute respiratory syndrome, cyanosis, myalgia, headache, encephalopathy, myocardial injury, heart failure, arrhythmia, coagulation dysfunction, acute kidney injury, confusion or inability to arouse, fatigue, and gastrointestinal symptoms.

In some embodiments, the subject has suffered trauma (e.g., a blunt force trauma or a penetrating trauma).

In some embodiments, the subject has suffered burn wounds.

In some embodiments, the subject has suffered Steven Johnson's syndrome.

In some embodiments, the subject is concurrently treated with assisted ventilation and oxygenation.

In some embodiments, the subject is concurrently treated with an anti-viral drug.

In some embodiments, the antiviral agent is remdesivir, chloroquine, hydroxychloroquine, baricitinib, lopinavir/ritonavir, interferon beta, umifenovir, favipiravir, tocilizumab, or ribavirin.

In some embodiments, the subject is concurrently treated with an anti-inflammatory drug.

In some embodiments, the anti-inflammatory agent is dexamethasone, celecoxib, diclofenac, difunisal, etodolac, ibuprofen, indomethacin, ketoprofen, ketorolac, nabumetone, naproxen, oxaprozin, prednisone, prednisolone, methylprednisolone, or metformin.

In some embodiments, the subject is concurrently treated with anti-coagulation drug.

In some embodiments, the anti-coagulation agent is tPA, heparin, low molecular weight heparin, or bivalirudin.

In some embodiments, the subject has or is at risk of developing acute respiratory distress syndrome, severe cardiovascular damage, acute cardiac injury, acute kidney injury, septic shock, multi-organ failure, and increased risk of death.

In some embodiments, the treatment of the invention reverses, alleviates, ameliorates, inhibits, slows down, or stops the progression or severity of one or more COVID-19 disease symptom, e.g., as described herein.

In yet another aspect, the disclosure features a method of treating a cancer in a subject in need thereof by administering a therapeutically effective amount of the compound disclosed herein, or a pharmaceutically acceptable salt thereof, or the pharmaceutical composition disclosed herein to the subject.

In some embodiments, the cancer is gastric cancer, colon cancer, melanoma, and multiple myeloma.

In another aspect, the invention can be used in the treatment of a subject having sepsis, bacterial infection, protozoan infection, fungal infection, viremia, bacteremia, protozoan infection, or fungal infection

### Definitions

The term “acyl,” as used herein, represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, represents a chemical substituent of formula —OR, where R is acyl. An optionally substituted acyloxy is an acyloxy that is optionally substituted as described herein for acyl.

The term “alkanoyl,” as used herein, represents a chemical substituent of formula —C(O)—R, where R is alkyl. An optionally substituted alkanoyl is an alknanoyl that is optionally substituted as described herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, two substituents combine to form a group -L-CO—R, where L is a bond or optionally substituted C₁-˜ alkylene, and R is hydroxyl or alkoxy. Each of the substituents may itself be unsubstituted or, valency permitting, substituted with unsubstituted substituent(s) defined herein for each respective group.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The expression “C_{x-y},” as used herein, indicates that the group, the name of which immediately follows the expression, when unsubstituted, contains a total of from x to y carbon atoms. If the group is a composite group (e.g., aryl alkyl), C_{x,y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkoxy,” as used herein, represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “halo,” as used herein, represents a halogen selected from bromine, chlorine, iodine, and fluorine.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic non-aromatic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{a}, where R^{a} is hydrogen, alkyl, aryl, arylalkyl, cycloalkyl, heterocyclyl, or heteroaryl; and —CON(R^{b})₂, where each R^{b} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH.

The term “oxo,” as used herein, represents a divalent oxygen atom (e.g., the structure of oxo may be shown as ═O).

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “protecting group,” as used herein, represents a group intended to protect a hydroxy, an amino, or a carbonyl from participating in one or more undesirable reactions during chemical synthesis.

The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “subject,” as used herein, represents a human or non-human animal (e.g., a mammal) that is suffering from a disease or condition, as determined by a qualified professional (e.g., a doctor or a nurse practitioner) with or without known in the art laboratory test(s) of sample(s) from the patient.

The term “treating,” as used in reference to a disease or a condition in a patient, is intended to refer to obtaining beneficial or desired results, e.g., clinical results, in a patient by administering a compound of the invention to the patient. Beneficial or desired results may include alleviation or amelioration of one or more symptoms of a disease or condition; diminishment of extent of a disease or condition; stabilization (i.e., not worsening) of a disease or condition; prevention of the spread of a disease or condition; delay or slowing the progress of a disease or condition; palliation of a disease or condition; and remission (whether partial or total). “Palliating” a disease or condition means that the extent and/or undesirable clinical manifestations of the disease or condition are lessened and/or time course of the progression is slowed, as compared to the extent or time course in the absence of the treatment with the compound of the invention. Non-limiting examples of diseases and conditions that may be treated using compounds disclosed herein are

### BRIEF DESCRIPTION OF THE DRAWINGS

FIG. 1A is a bar graph of HEK-Blue™ assay (HEK-IL-18 Sensor Cells): IL-18 activity assay for IL-18BP at different concentrations (NT, 1.5 nM, 7.5 nM, and 37.5 nM).

FIG. 1B is a bar graph of HEK-Blue™ assay (HEK-IL-18 Sensor Cells): IL-18 activity assay for compound 1 at different concentrations (NT, 1.5 nM, 7.5 nM, and 37.5 nM).

FIG. 1C is a bar graph of HEK-Blue™ assay (HEK-IL-18 Sensor Cells): IL-18 activity assay for compound 10 at different concentrations (NT, 1.5 nM, 7.5 nM, and 37.5 nM).

FIG. 1D is a bar graph of HEK-Blue™ assay (HEK-IL-18 Sensor Cells): IL-18 activity assay for compound 15 at different concentrations (NT, 1.5 nM, 7.5 nM, and 37.5 nM).

FIG. 2A is a bar graph of HEK-Blue SEAP reporter assay for IL-18BP at low dose range (800 pM, 200 pM, 50 pM, 12.5 pM, and NT).

FIG. 2B is a bar graph of HEK-Blue SEAP reporter assay for compound 1 at low dose range (800 pM, 200 pM, 50 pM, 12.5 pM, and NT).

FIG. 2C is a bar graph of HEK-Blue SEAP reporter assay for compound 10 at low dose range (800 pM, 200 pM, 50 pM, 12.5 pM, and NT).

FIG. 2D is a bar graph of HEK-Blue SEAP reporter assay for compound 15 at low dose range (800 pM, 200 pM, 50 pM, 12.5 pM, and NT).

FIG. 3A is a bar graph showing toxicity assay of compound 15 in Saphenous Vein Endothelial (EC) at different concentrations (10 nM, 100 nM, 1 μM, and 10 μM) and times (4, 28, and 76 hours).

FIG. 3B is a bar graph showing toxicity assay of compound 15 in Saphenous Vein Endothelial (EC) at different concentrations (10 nM, 100 nM, 1 μM, and 10 μM) and times (4, 28, and 76 hours).

FIG. 3C is a bar graph showing toxicity assay of compound 15 in Saphenous Vein Endothelial (EC) at different concentrations (10 nM, 100 nM, 1 μM, and 10 μM) and times (4, 28, and 76 hours).

FIG. 3D is a bar graph showing toxicity assay of compound 15 in Smooth Muscle Cells (SMC) at different concentrations (10 nM, 100 nM, 1 μM, and 10 μM) and times (4, 24, 28, 48, and 72 hours).

FIG. 3E is a bar graph showing toxicity assay of compound 15 in Smooth Muscle Cells (SMC) at different concentrations (10 nM, 100 nM, 1 μM, and 10 μM) and times (4, 24, 28, 48, and 72 hours).

FIG. 3F is a bar graph showing toxicity assay of compound 15 in Smooth Muscle Cells (SMC) at different concentrations (10 nM, 100 nM, 1 μM, and 10 μM) and times (24, 48, and 72 hours).

FIG. 4A is a bar graph depicting toxicity assay of compound 1 in Smooth Muscle Cells (SMC) at different concentrations (10 nM, 100 nM, 1 μM and 10 μM) and times (4, 24, 28, and 72 hours).

FIG. 4B is a bar graph depicting toxicity assay of compound 10 in Smooth Muscle Cells (SMC) at different concentrations (10 nM, 100 nM. 1 μM and 10 μM) and times (4, 24, 28, 72 hours).

FIG. 5 is a bar graph comparing Interferon-γ expression in NK92 cells of IL-18 and compounds 1V and 15.

FIG. 6 is a bar graph comparing Interferon-γ expression in NK92 cells of IL-18, compounds 1 and 1V.

FIG. 7 is a line graph comparing Microscale Thermophoresis (MST) high binding affinity events of ligands (compounds 1 (top and bottom lines) and 15 (middle line) with multiple affinities. FIG. 14 further depicts first phase of biphasic curves, occurring at low ligand concentrations. Higher ligand concentrations are removed from analysis and colored grey.

FIG. 8 is a line graph comparing MST low binding affinity events of ligands (compounds 1 (top and bottom lines) and 15 (middle line)) with multiple affinities. FIG. 15 further depicts second phase of biphasic curves, occurring at high ligand concentrations. Lower ligand concentrations are removed from analysis and colored grey.

FIG. 9A is a line graph comparing MST binding affinity events of BP (top line) and compound 1 (bottom line) with multiple affinities.

FIG. 9B is a line graph comparing MST binding affinity events of BP (bottom line) and compound 10 (top line) with multiple affinities.

FIG. 9C is a line graph comparing MST binding affinity events of BP (bottom line) and compound 15 (top line) with multiple affinities.

FIG. 10A is a bar graph showing cytokine levels in human saphenous vein segments incubated with a control, LPS alone, compound 1+LPS, compound 10+LPS, or compound 15+LPS.

FIG. 10B is a bar graph showing TNF-α levels in human saphenous vein segments incubated with a control, LPS alone, compound 1+LPS, compound 10+LPS, or compound 15+LPS.

FIG. 10C is a bar graph showing IL6 levels in human saphenous vein segments incubated with a control, LPS alone, compound 1+LPS, compound 10+LPS, or compound 15+LPS.

FIG. 10D is a bar graph showing IL8 levels in human saphenous vein segments incubated with a control, LPS alone, compound 1+LPS, compound 10+LPS, or compound 15+LPS.

### DETAILED DESCRIPTION

In general, the invention provides compounds as described herein, compositions including them, and methods of their use. The compounds and compositions disclosed herein may be useful in the treatment of an inflammatory or autoimmune disorder. For example, the compounds and compositions disclosed herein may be useful in the treatment of rheumatoid arthritis, osteoarthritis, lupus, psoriasis, inflammatory bowel disease, vascular graft failure (e.g., peripheral vascular graft failure, coronary artery graft failure, restenosis after stent graft, or restenosis after balloon angioplasty), heart disease (e.g., cardiomyopathy, congestive heart failure, or ischemic coronary heart disease), vascular disease (e.g., intimal hyperplasia, atherosclerosis, coronary artery disease, restenosis, primary hypertension, secondary hypertension, peripheral vascular disease, or critical limb-threatening ischemia), type 1 diabetes, type 2 diabetes, metabolic syndrome, diabetic wound healing, and kidney disease (e.g., chronic kidney disease, end-stage renal disease, or kidney failure). The compounds and compositions disclosed herein may also be useful in the treatment of cancer (e.g., gastric cancer, colon cancer, melanoma, or multiple myeloma).

Without wishing to be bound by theory, compounds disclosed herein may inhibit interleukin-18 (IL18) and thus may be used in the treatment of a disease or condition.

An IL18 inhibitor may be, e.g., a compound of formula (I):

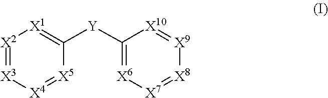

or a pharmaceutically acceptable salt thereof,
where:

each of X¹, X², X³, X⁴, X⁵, X⁶, X⁷, X⁸, X⁹, and X¹⁰ is independently CR¹ or N, provided that no more than two of X¹, X², X³, X⁴, and X⁵ is N, and provided that no more than two of X⁶, X⁷, X⁸, X⁹, and X¹⁰ is N;

Y is O, C(R²)₂, or S(O)ₜ;

each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R² is independently hydrogen, hydroxyl, or optionally substituted C₁-C₆alkyl; or both R² combine to form ═O, ═S, or ═NR⁵;

R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R⁴ is independently optionally substituted C₁-C₆ alkyl, optionally substituted C₆-C₁₀ aryl, optionally substituted heteroaryl, or optionally substituted heterocyclyl;

R⁵ is hydrogen, hydroxyl, —S(O)₂R⁴, or optionally substituted or C₁-C₆ alkyl; and

each t is independently 0, 1, or 2.

In some embodiments, the compound of formula (I) is not a compound selected from the group consisting of:

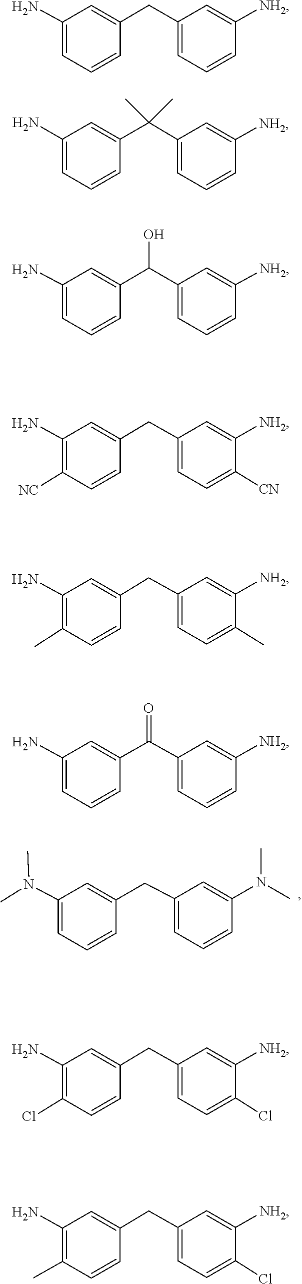

and pharmaceutically acceptable salts thereof.

In some embodiments, the IL18 inhibitor is a compound of formula (Ia):

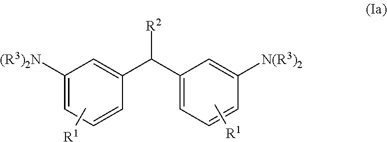

or a pharmaceutically acceptable salt thereof, wherein:

each R¹ is independently halogen, hydroxyl, cyano, nitro, —N(R³)₂, —S(O)ₜR⁴, —OR³, optionally substituted C₁-C₆ alkyl, optionally substituted C₆-C₁₀aryl, optionally substituted C₃-C₈cycloalkyl, or optionally substituted heteroaryl; and

the remaining variables are as described for formula (I).

In some embodiments, the IL18 inhibitor is a compound of formula (Ib):

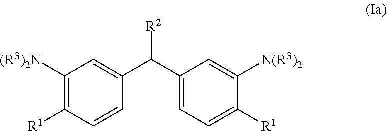

or a pharmaceutically acceptable salt thereof, wherein all variables are as described for formula (Ia).

An IL18 inhibitor may be, e.g., a compound of formula (II):

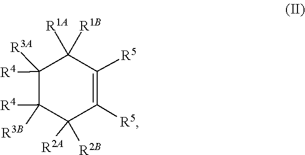

or pharmaceutically acceptable salts thereof, where:

(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or

(ii) each of R^{1A} and R^{2A} is independently hydrogen, halogen, hydroxyl, cyano, nitro, —S(O)ₜR⁶, or —OR⁷; R^{1B} and R^{3A}, together with the atoms to which they are attached, combine to form a double bond; and R^{2B} and R^{3B}, together with the atoms to which they are attached, combine to form a double bond;

both R⁴, together with the atoms to which they are attached, combine to form an optionally substituted heterocyclic ring;

both R⁵, together with the atoms to which they are attached, combine to form an optionally substituted heterocyclic ring;

each R⁶ is independently hydrogen, halogen, cyano, or optionally substituted C₁-C₆ alkyl; and

each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl; and

each t is independently 0, 1, or 2.

In some embodiments, the compound of formula (II) is not a compound selected from the group consisting of:

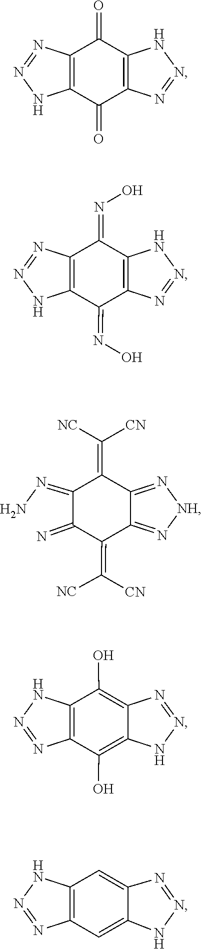

and pharmaceutically acceptable salts thereof.

In some embodiments, the IL18 inhibitor is compound of formula (IIa):

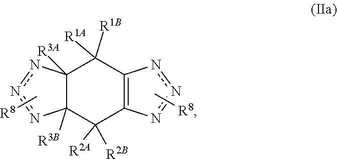

or a pharmaceutically acceptable salts thereof, wherein:

each R⁸ is independently H or optionally substituted C₁-C₆ alkyl; and

the remaining variables are as described for formulas (II).

An IL18 inhibitor may be, e.g., a compound of formula (III):

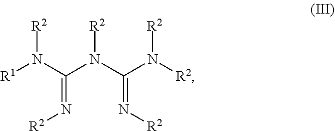

or a pharmaceutically acceptable salt thereof, where:

R¹ is an optionally substituted C₆-C₁₀ aryl or optionally substituted heteroaryl; and

each R² is independently hydrogen or an optionally substituted C₁-C₆ alkyl.

In some embodiments, the compound of formula (III) is not a compound selected from the group consisting of:

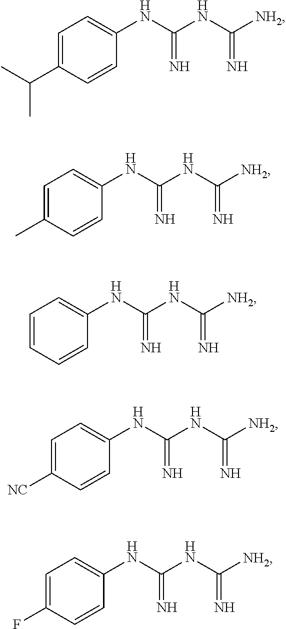 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]

and pharmaceutically acceptable salts thereof.

In some embodiments, the IL18 inhibitor is a compound of formula (IIIa):

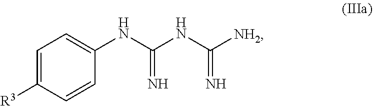

or a pharmaceutically acceptable salt thereof,

where:

R³ is optionally substituted C₁-C₆ alkyl, halogen, —OR⁴, —S(O)ₜR⁵, or —N(R⁴)₂;

each R⁴ is independently hydrogen or optionally substituted C₁-C₆ alkyl;

R⁵ is optionally substituted C₁-C₆ alkyl, optionally substituted C₆-C₁₀ aryl, or optionally substituted heteroaryl.

Exemplary compounds are shown in Table 1.

TABLE 1

| | |
|---|---|
| 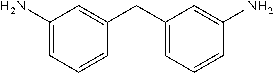 [SMILES: Nc1cccc(Cc2cccc(N)c2)c1] | 1 |
| 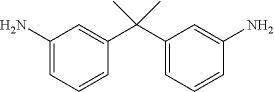 [SMILES: CC(C)(c1cccc(N)c1)c1cccc(N)c1] | 2 |
| 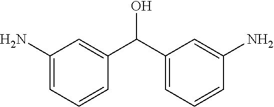 [SMILES: Nc1cccc(C(O)c2cccc(N)c2)c1] | 3 |
| 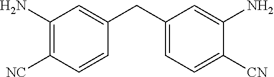 [SMILES: N#Cc1ccc(Cc2ccc(C#N)c(N)c2)cc1N] | 4 |
| 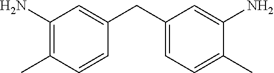 [SMILES: Cc1ccc(Cc2ccc(C)c(N)c2)cc1N] | 5 |
| 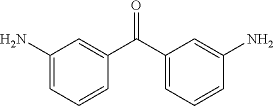 [SMILES: Nc1cccc(C(=O)c2cccc(N)c2)c1] | 6 |
| 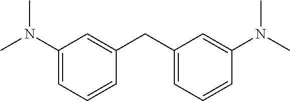 | 7 |
| 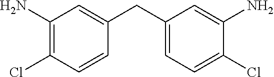 [SMILES: Nc1cc(Cc2ccc(Cl)c(N)c2)ccc1Cl] | 8 |
| 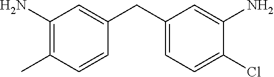 [SMILES: Cc1ccc(Cc2ccc(Cl)c(N)c2)cc1N] | 9 |
| 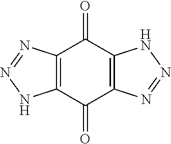 [SMILES: O=C1c2nn[nH]c2C(=O)c2nn[nH]c21] | 10 |
| 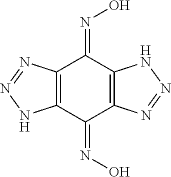 [SMILES: O/N=C1/c2nn[nH]c2/C(=N/O)c2nn[nH]c21] | 11 |
| 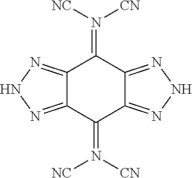 | 12 |
| 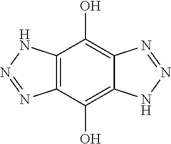 [SMILES: Oc1c2nn[nH]c2c(O)c2nn[nH]c12] | 13 |
| 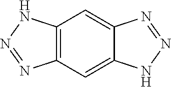 [SMILES: c1c2nn[nH]c2cc2nn[nH]c12] | 14 |
| 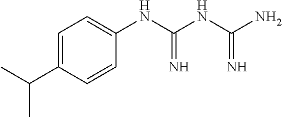 [SMILES: CC(C)c1ccc(NC(=N)NC(=N)N)cc1] | 15 |
| 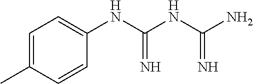 | 16 |
| 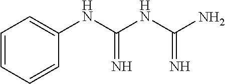 [SMILES: N=C(N)NC(=N)Nc1ccccc1] | 17 |
| 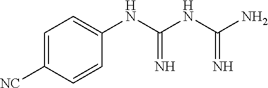 [SMILES: N#Cc1ccc(NC(=N)NC(=N)N)cc1] | 18 |
| 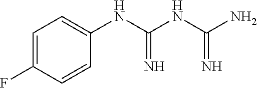 [SMILES: N=C(N)NC(=N)Nc1ccc(F)cc1] | 19 |

or a pharmaceutically acceptable salt of any of these compounds.

A non-limiting example of a pharmaceutically acceptable salt of a compound disclosed herein is:

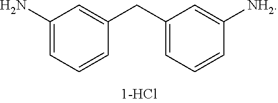 [SMILES: Nc1cccc(Cc2cccc(N)c2)c1]

A non-limiting example of a pharmaceutically acceptable salt of a compound disclosed herein is:

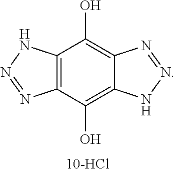 [SMILES: Oc1c2nn[nH]c2c(O)c2nn[nH]c12]

A non-limiting example of a pharmaceutically acceptable salt of a compound disclosed herein is:

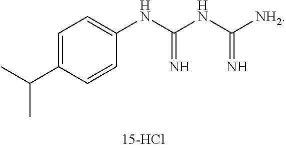 [SMILES: CC(C)c1ccc(NC(=N)NC(=N)N)cc1]

### Methods of Use

The compounds disclosed herein may be used to inhibit the interaction between IL18 and IL18R in a medium (e.g., a tissue or bodily fluid of a subject) including IL18 and a cell expressing IL18R. The methods typically include the step of contacting a compound disclosed herein with the cell in the medium (by, e.g., administration to a subject including the cell). The contacting step may result in inhibition of interaction between IL18 and IL18R.

The compounds disclosed herein may be used in methods of treating an inflammatory or autoimmune disorder in a subject in need thereof. The methods typically include the step of administering a therapeutically effective amount of the compounds disclosed herein or a pharmaceutically acceptable salt thereof to the subject. The compound or a pharmaceutically acceptable salt thereof may be administered to the subject as a pharmaceutical composition. The methods may be used to treat, e.g., rheumatoid arthritis, osteoarthritis, lupus, psoriasis, inflammatory bowel disease, vascular graft failure (e.g., peripheral vascular graft failure, coronary artery graft failure, restenosis after stent graft, or restenosis after balloon angioplasty), heart disease (e.g., cardiomyopathy, congestive heart failure, or ischemic coronary heart disease), vascular disease (e.g., intimal hyperplasia, atherosclerosis, coronary artery disease, restenosis, primary hypertension, secondary hypertension, peripheral vascular disease, or critical limb-threatening ischemia), type 1 diabetes, type 2 diabetes, metabolic syndrome, diabetic wound healing, and kidney disease (e.g., chronic kidney disease, end-stage renal disease, or kidney failure). Alternatively, the methods may be used to treat, e.g., cancer (e.g., gastric cancer, colon cancer, melanoma, or multiple myeloma), trauma, burn wounds, or Steven Johnsons Syndrome.

The compounds described herein may be used in the treatment of an infection (e.g., a viral infection, such as a beta-coronavirus infection or an influenza infection). The compounds described herein may be used in the treatment of a viral infection, bacterian infection, protozoan infection, or fungal infection. The subject may be suffering from, e.g., acute respiratory distress syndrome (ARDS) (e.g., ARDS associated with an infection (e.g., a viral infection, such as a beta-coronavirus infection or an influenza infection)). The compounds described herein may be used, e.g., to treat a cytokine storm or endothelialitis (e.g., vascular endothelialitis). Infections (e.g., beta-coronavirus infections (e.g., SARS-CoV-2)) may lead to severe vascular dysfunction (e.g., vascular endothelialitis) and/or thromboinflammation. Eventually, the infections (e.g., beta-coronavirus infections (e.g., SARS-CoV-2)) may lead, e.g., to microvascular thrombi, which, in turn may lead to lung failure. The cytokine storm or endotheliitis may be, e.g., associated with an infection (e.g., a viral infection (e.g., a beta-coronavirus infection)). The infection may be, e.g., SARS-CoV-2, SARS-CoV, or MERS-CoV. Preferably, the infection is SARS-CoV-2. The subject may be, e.g., one that has been diagnosed with SARS-CoV-2 infection. The subject may have, e.g., COVID-19. The subject may be, e.g., an asymptomatic carrier of a beta-coronavirus (e.g., SARS-CoV-2). Alternatively, the subject may have one or more symptoms of COVID-19, e.g., fever, sore throat, runny nose, sneezing, nasal congestion, snoring, coughing, dry cough, shortness of breath, difficulty breathing, persistent pain or pressure in the chest, dyspnea, pneumonia, acute respiratory syndrome, cyanosis, myalgia, headache, encephalopathy, myocardial injury, heart failure, arrhythmia, coagulation dysfunction, acute kidney injury, confusion or inability to arouse, fatigue, or gastrointestinal symptoms. The subject may have or may be at risk of, e.g., developing acute respiratory distress syndrome, severe cardiovascular damage, acute cardiac injury, acute kidney injury, septic shock, multi-organ failure, or increased risk of death. The compounds and methods of the invention may reverse, alleviate, ameliorate, inhibit, slow down, or stop the progression or severity of one or more COVID-19 disease symptom, e.g., as described herein.

The subject may also be treated with, e.g., assisted ventilation or oxygenation. For example, the subject is concurrently treated with assisted ventilation and oxygenation. The subject may also be treated with an anti-viral drug. For example, the subject is concurrently treated with an anti-viral drug. Non-limiting examples of anti-viral drugs include remdesivir, chloroquine, hydroxychloroquine, baricitinib, lopinavir/ritonavir, interferon beta, umifenovir, favipiravir, tocilizumab, and ribavirin. The subject may also be treated with an anti-inflammatory drug. For example, the subject is concurrently treated with an anti-inflammatory drug. Non-limiting examples of anti-inflammatory drugs include dexamethasone, celecoxib, diclofenac, difunisal, etodolac, ibuprofen, indomethacin, ketoprofen, ketorolac, nabumetone, naproxen, oxaprozin, prednisone, prednisolone, methylprednisolone, and metformin. The subject may also be treated with an anti-coagulation drug. For example, the subject is concurrently treated with an anti-coagulation drug. Non-limiting examples of anti-coagulation drugs include, tPA, heparin, low molecular weight heparin, bivalirudin, aspirin.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

### Methods of Preparing Compounds

The compounds disclosed herein can be prepared by processes analogous to those established in the art, for example, by the reaction sequence shown in scheme 1. The numbering system used for the general schemes does not necessarily correspond to that employed elsewhere in the description or in the claims.

As shown in scheme 1, one strategy to access compound (3A) is to subject compound (2A) to alkylation, acylation, or sulfonylation reaction conditions. Compounds of invention (2A) may be obtained in two steps from compounds of the invention (1A) first nitration reaction, followed with the reduction of the nitro groups.

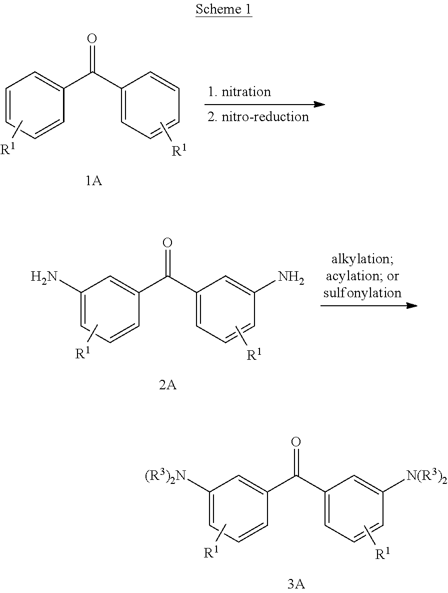

Compound (1A) may be prepared using techniques and methods known in the art (Scheme 2), e.g. oxidation of compound 5A, a Friedel-Crafts alkylation reaction product of compound 4A and compound 4B, or Friedel-Crafts acylation reaction of compound 4A and acyl chloride 6A using strong Lewis acids (e.g. AlCl₃, FeCl₃ or MXₙ reagents). Friedel-Crafts alkylation reactions utilizing benzyl alcohol as an electrophile proceed via a benzylic carbocation formed in situ from its ionization in the presence of an acid having strong ionizing and poor nucleophilic properties (e.g. TFA).

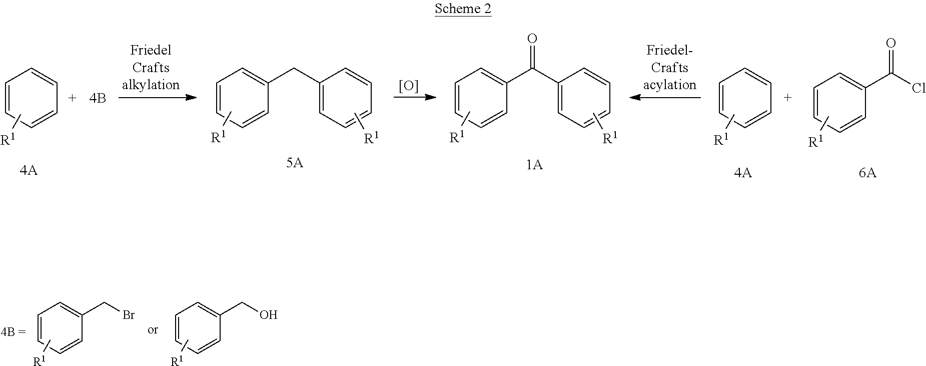

As shown in scheme 3, compounds of invention (8A) may be accessed using techniques and method known in the art, e.g., reduction of carbonyl moiety to alkane. Non-limiting examples of the reduction reactions include Clemmensen reduction and Wolff-Kishner reduction. Typically, Clemmensen reduction utilizes zinc (e.g., zinc amalgam) under acidic conditions (e.g., conc. aq. HCl). Wolff-Kishner reduction typically involves thermolysis of hydrazones obtained from hydrazine and a ketone (e.g., compound 2A). Compound (8A) may be directly obtain from the reduction of the carbonyl moiety in compound (3A) or alternatively through alkylation, acylation or sulfonylation of compound of invention (7A), which in turn may be access upon reduction of ketone moiety in compound (2A).

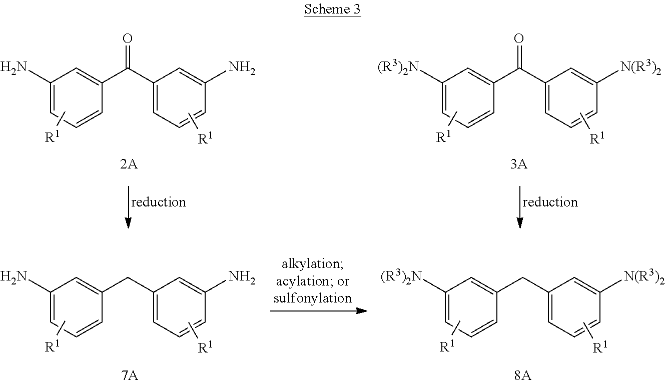

One skilled in the art would know that the carbonyl moiety in compound (3A) may be functionalized using appropriate reaction conditions to allow the preparation of compounds of invention (9A-10A and 12A-13A), scheme 4: condensation with an amine may give compound of the invention (9A); reaction with mild thionating reagents such as Lawesson's reagent may yield compound of the invention (10A); Grignard reaction may generate secondary alcohol 30, which may be activated into a good leaving group (e.g. O-triflate, I or Br) and subsequently reacted with a nucleophile to provide compound of the invention (12A); 1,2-reduction (e.g. using sodium borohydride as a reducing agent) may provide compound of the invention (13A).

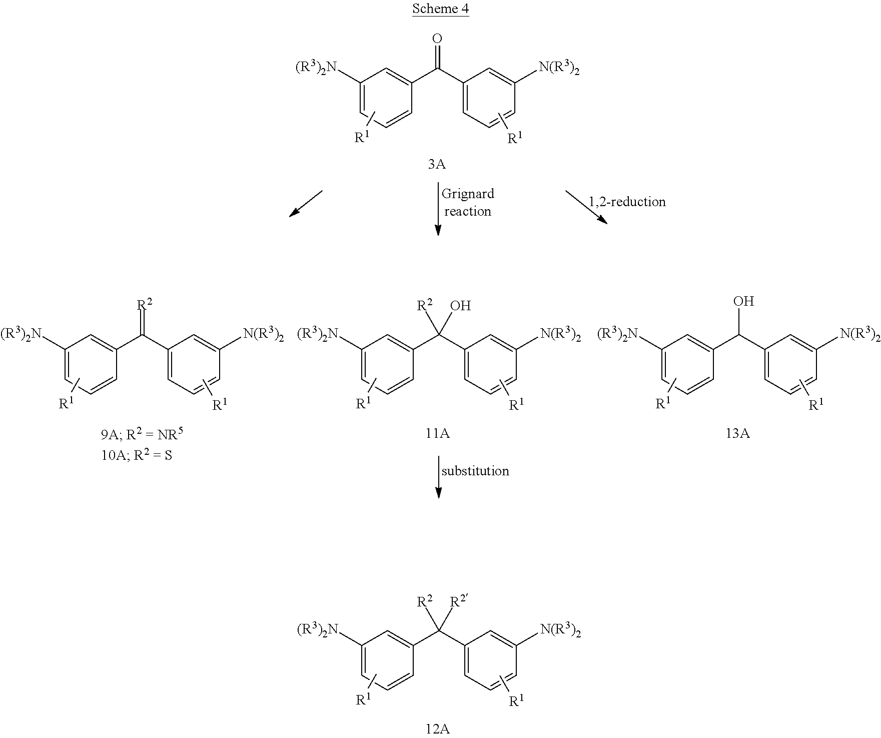

Compounds of the invention (18A) may be prepared via alkylation, acylation or sulfonylation of compound (17A). Compound (17A) may be obtained from the reduction of the nitro groups in compound (16A), which is the oxidative product of the sulfur moiety in compound (15A). Condensation of compound 14A with sodium sulfide may provide compound (15A).

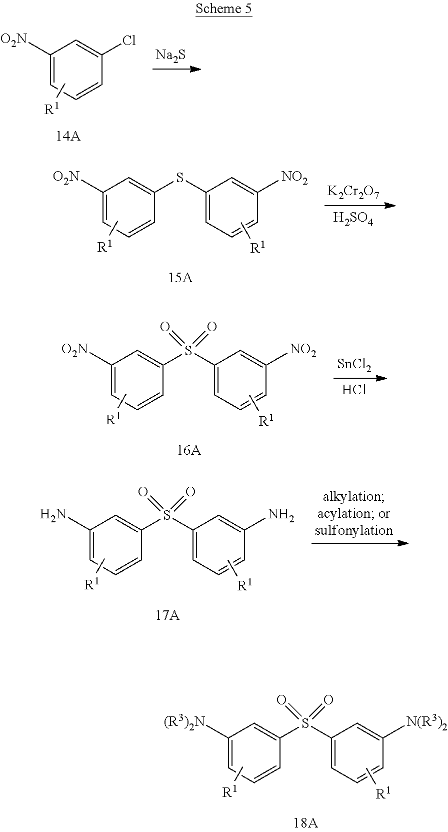

One method to access compounds of the invention (23A) is through the alkylation, acylation or sulfonylation of compound (22A), scheme 6. Reduction of the nitro group in compound (21A) (e.g. using SnCl₂) may result in compound (22A). This in turn may be obtain from O-arylation reaction of compound 19A and compound 20A.

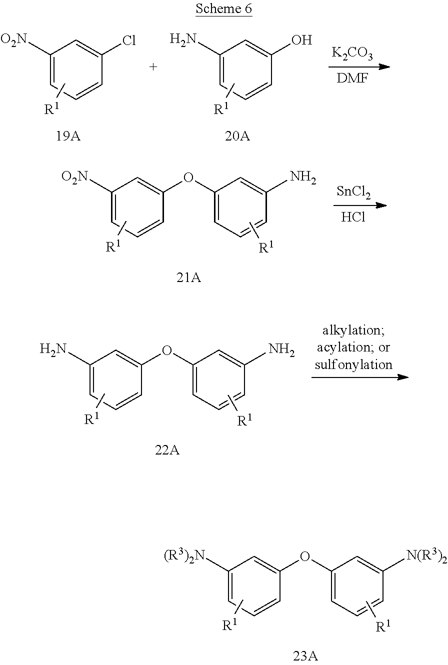

It is known in the art that dipolar cycloaddition reaction of compound 24A and azides may yield compounds of the invention (25A), scheme 7. Subjecting compound (25A) under applicable reaction conditions may give access to respective compounds of this invention: reaction with amines may yield compounds of the invention (26A); reacting with Lawesson's reagent may give compounds of the invention (27A); may be converted to compounds of the invention (28A) by reacting with ammonium hydroxide in the presence of pyridine in ethanol; Knoevenagel condensation reaction with malononitrile in the presence of a base may result in compounds of the invention (29A).

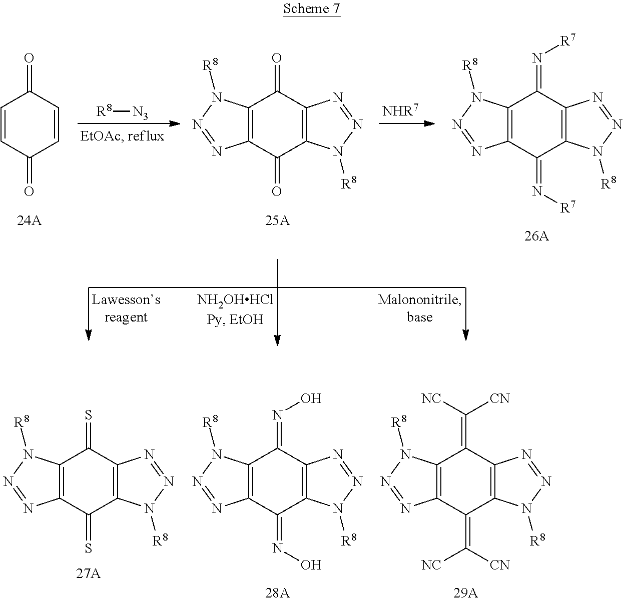

One option to obtain compound of the invention (31A) as shown in scheme 8 is to react compound 30A with sodium nitrite in the presence of bromic acid. Alkylation reaction following standard protocols (e.g., using base such as K₂CO₂, Cs₂CO₃) may yield compound (32A), an advance intermediate to prepare other compounds of this invention: copper-catalyzed Ullmann amination reaction conditions may provide compounds of the invention (33A) (e.g., compound (33A), where R⁷ is H may be further reacted under Sandmeyer reaction conditions to give compounds of the invention (34A)); Grignard reaction may give compounds of the invention (54A); Pd-catalyzed nitration reaction may allow access to compounds of the invention (36A).

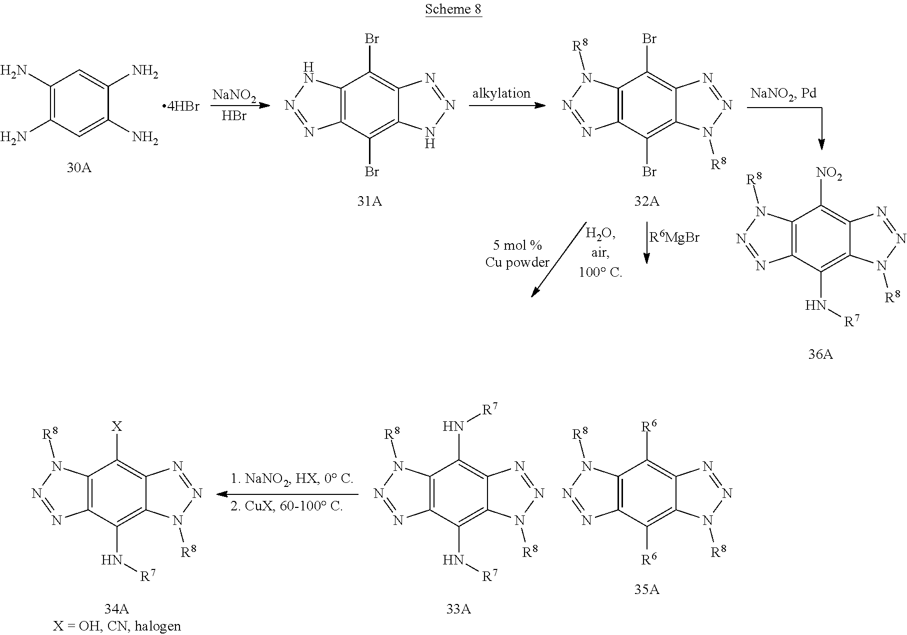

Compounds of the invention (39A) may be achieved from NMP-catalyzed condensation of compound 37A and compound 38A (Scheme 9). Bromination of compound (39A) may give compound (40A), which may be subjected to proper reaction conditions as describe in scheme 8 to access compounds of the invention (41A), (42A), (43A), and (44A).

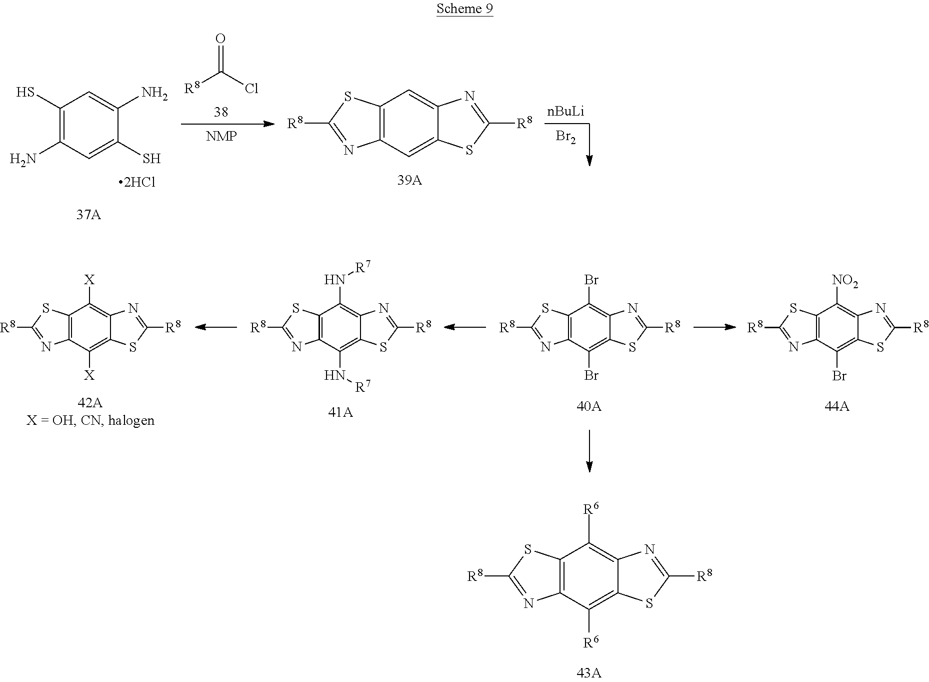

One approach to access compounds of the invention (47A) is the condensation of compound 45A and compound 46A (e.g., using tosylic acid monohydrate and copper iodide as co-catalyst) (Scheme 10). Bromination of compound (47A) may provide compound (48A), which when subjected to appropriate reaction conditions as describe in scheme 8 may give access to compounds of the invention (49A), (50A), (51A), and (52A).

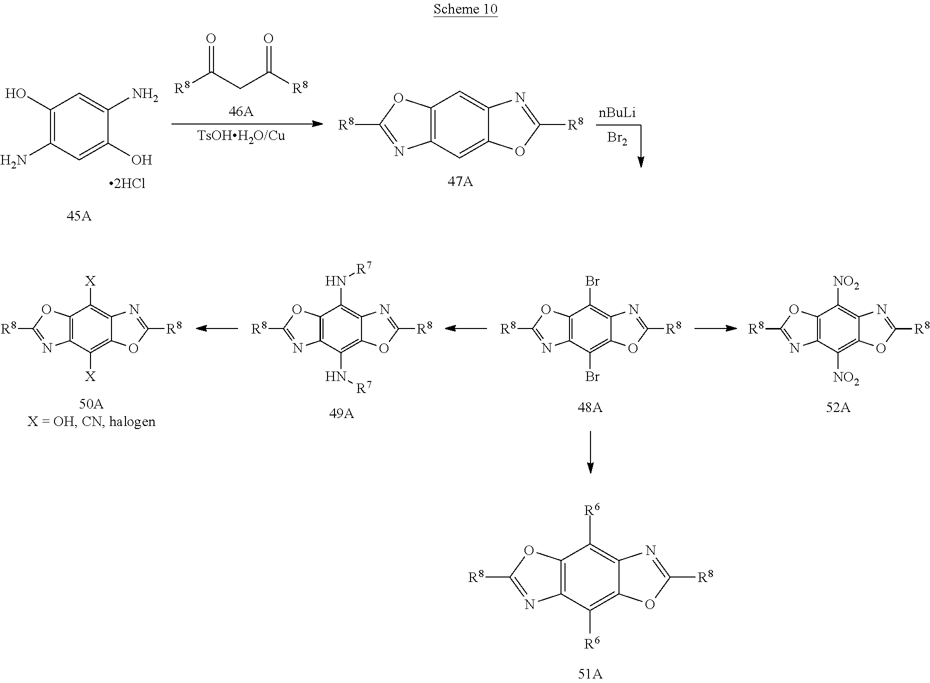

As shown in scheme 11, one strategy to synthesize compounds of the invention (55A) is to utilize known procedures in the art of amine 54A nucleophilic addition to electrophile (dicyandiamide 53A). To facilitate access to more substituted compounds of invention (61A), dicyandiamide derivatives 60A may reacted with amine 54A as shown in scheme 12.

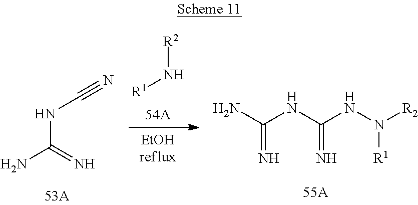

Compound 60A may be prepared according to known procedures in the art. The reaction of amine 56A with isothiocynate 57A (prepared from reacting carbon disulfide with amine 56A) may yield thiourea 58A, which upon reacting with methyl iodide may give salt 59A. Desulfurylation of compound 59A with cyanamide may yield compound 60A.

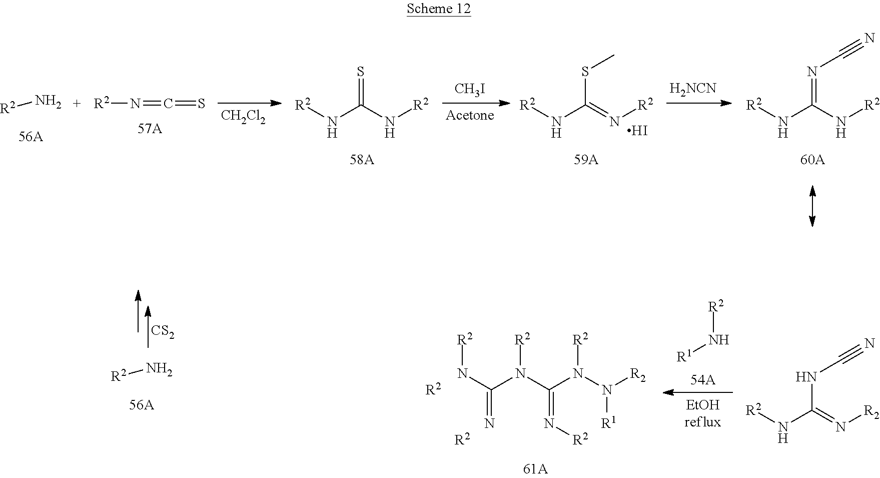

In the reactions described above, it may be necessary to protect reactive functional groups (e.g., hydroxyl, amino, thio, or carboxyl groups) to avoid their unwanted participation in the reactions. The incorporation of such groups, and the methods required to introduce and remove them are known to those skilled in the art (for example, Greene). The deprotection step may be the final step in the synthesis such that the removal of protecting groups affords compounds of formula (I), (Ia), (Ib), (II), (IIa), (III), or (IIIa) as disclosed herein. Starting materials used in any of the schemes above can be purchased or prepared by methods described in the chemical literature, or by adaptations thereof, using methods known by those skilled in the art. The order in which the steps are performed can vary depending on the groups introduced and the reagents used, but would be apparent to those skilled in the art.

Compounds of any of formula (I), (Ia), (Ib), (II), (IIa), (III), or (IIIa), or any of the intermediates described in the schemes above, can be further derivatized by using one or more standard synthetic methods describe herein or known to those skilled in the art. Such methods can involve substitution, oxidation or reduction reactions. These methods can also be used to obtain or modify compounds of formula (I), (Ia), (Ib), (II), (IIa), (III), or (IIIa), or any preceding intermediates by modifying, introducing or removing appropriate functional groups. Particular substitution approaches include alkylation, arylation, heteroarylation, acylation, thioacylation, halogenation, sulfonylation, nitration, formylation, hydrolysis, and coupling procedures. These procedures can be used to introduce a functional group onto the parent molecule (e.g., the nitration or sulfonylation of aromatic rings) or to couple two molecules together (for example to couple an amine to a carboxylic acid to afford an amide; or to form a carbon-carbon bond between two heterocycles). For example, alcohol or phenol groups can be converted to ether groups by coupling a phenol with an alcohol in a solvent, e.g., tetrahydrofuran in the presence of a phosphine (e.g., triphenylphosphine) and a dehydrating agent (e.g., diethyl-, diisopropyl-, or dimethylazodicarboxylate). Alternatively, ether groups can be prepared by deprotonation of an alcohol, using a suitable base (e.g., sodium hydride) followed by the addition of an alkylating agent (e.g., an alkyl halide or an alkylsulfonate).

In another example, —OH groups may be generated from the corresponding ester, acid, acid chloride or aldehyde by reduction with a suitable reducing agent, e.g., a complex metal hydride, e.g., lithium aluminum hydride in a solvent (e.g., tetrahydrofuran).

In another example, hydroxyl groups (including phenolic OH groups) can be converted into leaving groups, e.g., halogen atoms or sulfonyloxy groups (e.g., alkylsulfonyloxy, e.g., trifluoromethylsulfonyloxy, or arylsufonyl, e.g., p-toluenesulfonyloxy) using conditions known to those skilled in the art. For example, an aliphatic alcohol can be reacted with thionyl chloride in a halogenated hydrocarbon (e.g., dichloromethane) to afford the corresponding alkylchloride. A base (e.g., triethylamine) can also be used in the reaction.

In another example, ester groups can be converted to the corresponding carboxylic acid by acid- or base-catalyzed hydrolysis depending on the nature of the ester group. Acid catalysed hydrolysis can be achieved by treatment with an organic or inorganic acid (e.g., trifluoroacetic acid in an aqueous solvent, or a mineral acid, e.g., hydrochloric acid in a solvent, e.g., dioxan). Base catalyzed hydrolysis can be achieved by treatment with an alkali metal hydroxide (e.g., lithium hydroxide in an aqueous alcohol, e.g., methanol).

In another example, aromatic halogen substituents in the compounds may be subjected to halogen-metal exchange by treatment with a base (e.g., a lithium base, e.g., n-butyl or t-butyl lithium) optionally at a low temperature (e.g., −78° C.) in a solvent (e.g., tetrahydrofuran) and the mixture may then quenched with an electrophile to introduce a desired substituent. Thus, for example, a formyl group can be introduced by using dimethylformamide as the electrophile. Aromatic halogen substituents can also be subjected to palladium catalyzed reactions to introduce groups, e.g., carboxylic acids, esters, cyano, or amino substituents.

In another example, aromatic halogen substituents in the compounds may participate in a range of metal catalyzed reactions to introduce alternative functional groups, e.g., amines, amides, ethers, thiols, aryl groups, or heteroaryl groups.

Particular oxidation approaches include dehydrogenations and aromatization, and the addition of oxygen to certain functional groups. For example, aldehyde groups can be prepared by oxidation of the corresponding alcohol using conditions well known to those skilled in the art. For example, an alcohol can be treated with an oxidizing agent (e.g., the Dess-Martin reagent) in a solvent (e.g., a halogenated hydrocarbon, for example dichloromethane). Alternative oxidizing conditions can be used, e.g., treatment with oxalyl chloride and an activating amount of dimethylsulfoxide and subsequent quenching by the addition of an amine (e.g., triethylamine). Such a reaction can be carried out in an appropriate solvent (e.g., a halogenated hydrocarbon, for example dichloromethane) and under appropriate conditions (e.g., cooling below room temperature, e.g., to −78° C. followed by warming to room temperature). In another example, sulphur atoms can be oxidized to the corresponding sulfoxide or sulfone using an oxidizing agent (e.g., a peroxy acid, e.g., 3-chloroperoxybenzoic acid) in an inert solvent (e.g., a halogenated hydrocarbon, e.g., dichloromethane) at around ambient temperature.

Particular reduction approaches include the removal of oxygen atoms from particular functional groups, saturation (or partial saturation) of unsaturated compounds including aromatic rings. For example, primary alcohols can be generated from the corresponding ester or aldehyde by reduction, using a metal hydride (e.g., lithium aluminum hydride or sodium borohydride in a solvent, e.g., methanol).

Alternatively, —OH groups can be generated from the corresponding carboxylic acid by reduction, using a metal hydride (e.g., lithium aluminum hydride in a solvent, e.g., tetrahydrofuran). In another example, a nitro group may be reduced to an amine by catalytic hydrogenation in the presence of a metal catalyst (e.g., palladium on a solid support, e.g., carbon) in a solvent (e.g., an ether, e.g., tetrahydrofuran, or an alcohol, e.g., methanol), or by chemical reduction using a metal (e.g., tin or iron) in the presence of an acid (e.g., hydrochloric acid). In a further example an amine can be obtained by reduction of a nitrile, e.g., by catalytic hydrogenation in the presence of a metal catalyst (e.g., palladium on a solid support, e.g., carbon), or Raney nickel in a solvent (e.g., tetrahydrofuran) and under suitable conditions (e.g., cooling to below room temperature, e.g., to −78° C., or heating, e.g., to reflux).

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21^{st} Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2^{nd}, 3^{rd}, 4^{th}, 5^{th}, or 6^{th} day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted overtime according to the individual need and the professional judgment of the person administering or supervising the administration of the compounds or compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments doses of the compounds of the invention are administered to a patient over a time period that is 1-7 days; 1-12 weeks; or 1-3 months. In other embodiments, the compounds are administered to the patient over a time period that is, for example, 4-11 months or 1-30 years. In other embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a bacterial infection. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Buccal Administration

Dosages for buccal or sublingual administration typically are 0.1 to 500 mg per single dose as required. In practice, the physician determines the actual dosing regimen which is most suitable for an individual patient, and the dosage varies with the age, weight, and response of the particular patient. The above dosages are exemplary of the average case, but individual instances exist wherein higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a compound of the invention with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of compounds of the invention. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., Biesalski, U.S. Pat. No. 5,112,598 and Biesalski, U.S. Pat. No. 5,556,611, each of which is herein incorporated by reference).

### Formulations for Nasal or Inhalation Administration

The compounds may also be formulated for nasal administration. Compositions for nasal administration also may conveniently be formulated as aerosols, drops, gels, and powders. The formulations may be provided in a single or multidose form. In the case of a dropper or pipette, dosing may be achieved by the patient administering an appropriate, predetermined volume of the solution or suspension. In the case of a spray, this may be achieved, for example, by means of a metering atomizing spray pump.

The compounds may further be formulated for aerosol administration, particularly to the respiratory tract by inhalation and including intranasal administration. The compound will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the compound in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form comprises an aerosol dispenser, it will contain a propellant, which can be a compressed gas, e.g., compressed air or an organic propellant, e.g., fluorochlorohydrocarbon. The aerosol dosage forms can also take the form of a pump-atomizer.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils.

Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21^{st} Ed., Gennaro, Ed., Lippincott Williams & Wilkins (2005) and in the United States Pharmacopeia: the National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

The following examples are meant to illustrate the invention. They are not meant to limit the invention in any way.

### EXAMPLES

### Example 1. In Vitro Assays of the Compounds

Small Inhibitor Compound Preparation

Inhibitor compounds were solubilized and stored in DMSO at stock concentrations between 2.5-250 mM. Structure of compounds assayed are shown in Table 2.

TABLE 2

| | |
|---|---|
| 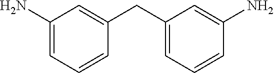 [SMILES: Nc1cccc(Cc2cccc(N)c2)c1] | 1 |
| 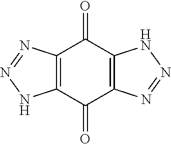 [SMILES: O=C1c2nn[nH]c2C(=O)c2nn[nH]c21] | 10 |
| 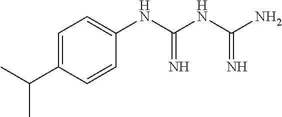 [SMILES: CC(C)c1ccc(NC(=N)NC(=N)N)cc1] | 15 |
| 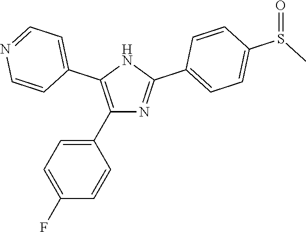 [SMILES: CS(=O)c1ccc(-c2nc(-c3ccc(F)cc3)c(-c3ccncc3)[nH]2)cc1] | 1V |

HEK-Blue IL18 Cell Reporter Assay

HEK-Blue IL18 Cells (Invivogen California, USA), are a derivative of HEK293 cells that have been stably transfected with IL18Rα/IL18RAP genes and a SEAP reporter gene that is expressed and secreted in specific response to IL18. Cells were grown in 25 mm² tissue culture flasks with DMEM culture medium containing 4.5 g/L glucose, 10% (v/v) heat-inactivated fetal bovine serum, 50 μ/mL penicillin, 50 μg/mL streptomycin, 100 μg/mL Normocin and 2 mM L-glutamine at 37° C. in 5% CO₂.

Stock concentration of IL18 was 750 μM stored in Tris-HCl buffer at −80° C. For treatment use, compounds were diluted in sterile PBS to 100 times the intended treatment concentration and administered into wells containing 198 μl media at volumes of 2 μl. Total DMSO contents for all vehicles was less than 0.02% prior to treatment, and less than 0.0002% within wells following treatment.

HEK-Blue IL18 cells were transferred from flasks and seeded at 40-50% confluency on 96-well culture plates 6 hours prior to treatment.

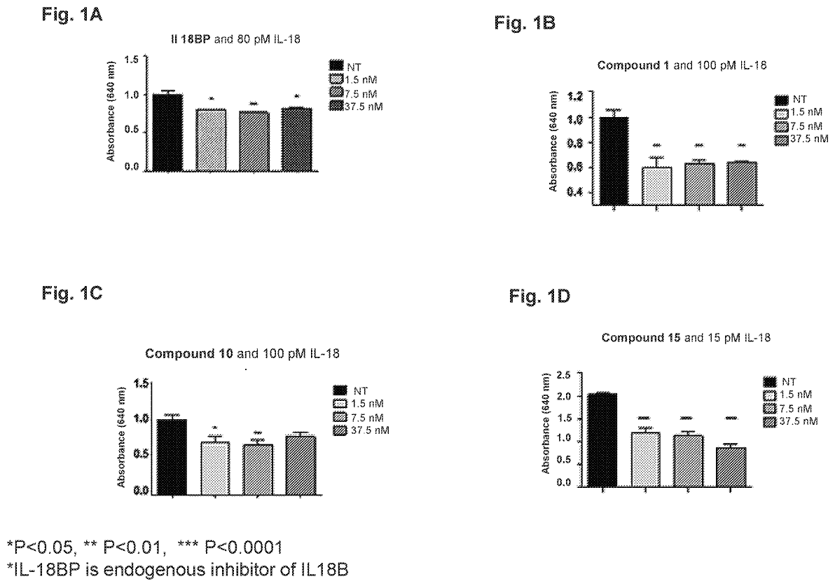

Phase I: Cells were treated with compounds at concentrations of 3.75 nM, 7.5 pM, and 1.5 pM, with three replicate wells for each concentration, and six replicate wells with no compound treatment (NT). All wells were then immediately co-treated with recombinant human IL18 at one concentration between 15 pM-100 pM. Following treatments, cells were incubated at 37° C. in 5% CO₂ for 18 hours. Cell confluency typically reached 70-80% (˜80,000-100,000 cells per well) by this point, 24 hours after seeding. IL18BP and compounds 1, 10, and 15 all significantly reduce IL-18 activity, see FIGS. 1A-1D respectively.

Phase II: Six Most potent inhibitory compounds were tested with the following protocol: six replicate wells treated with a selected compound at a low dose range (800 pM, 200 pM, 50 pM, 12.5 pM, NT, plus 50 pM IL18 co-treatment) and a higher dose range (1600 pM, 400 pM, 100 pM, 25 pM, NT, plus 100 pM IL18 treatment).

To detect SEAP activity, 20 μL supernatant from each well was transferred and mixed with 180 μl alkaline phosphatase detection medium (QUANTi-Blue, InvivoGen, California, USA), in a separate 96-well plate and incubated for 3 hours at room temperature. Supernatant SEAP activity was measured by reading the optical density (OD) at 640 nm using a spectrophotometer. OD values were standardized to NT mean=1.

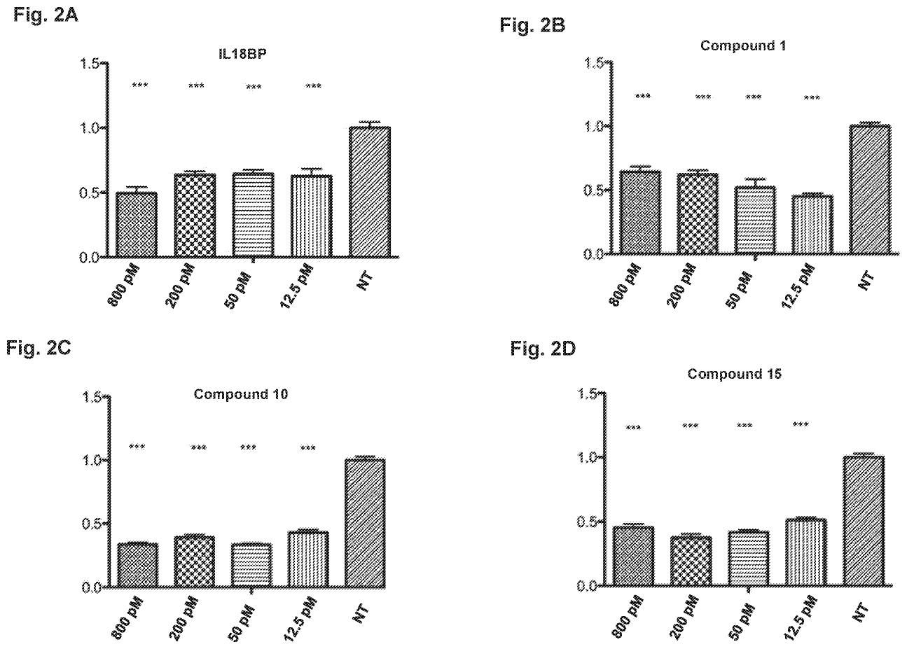

Statistical analysis was performed GraphPad Prism (California, USA) software. One-Way analysis of variance (ANOVA) with Dunnett's post-hoc analysis was used to determine significant variation between treatment and control (NT) groups treated only with IL18. IL18BP and compounds 1, 10, and 15 induction of SEAP production results are shown in FIGS. 2A-2D respectively.

Alamar Blue Assay for Cell Toxicity and Proliferation

Compounds that tested positive in the HEK Blue Assay were subjected to cell toxicity and proliferation assay using Alamar Blue. Primary human saphenous vein smooth muscle (SMC) and endothelial cells (EC) were left untreated or treated with inhibitor compounds (10 nM-10 μM) for a period of 3-4 days. Cell proliferation was measured using our previously published Alamar Blue Assay (Monahan et al., FASEB J., 23:557-564, 2009; Yoshida et al., J. Am. Coll. Surg. 213:668-67, 2011).

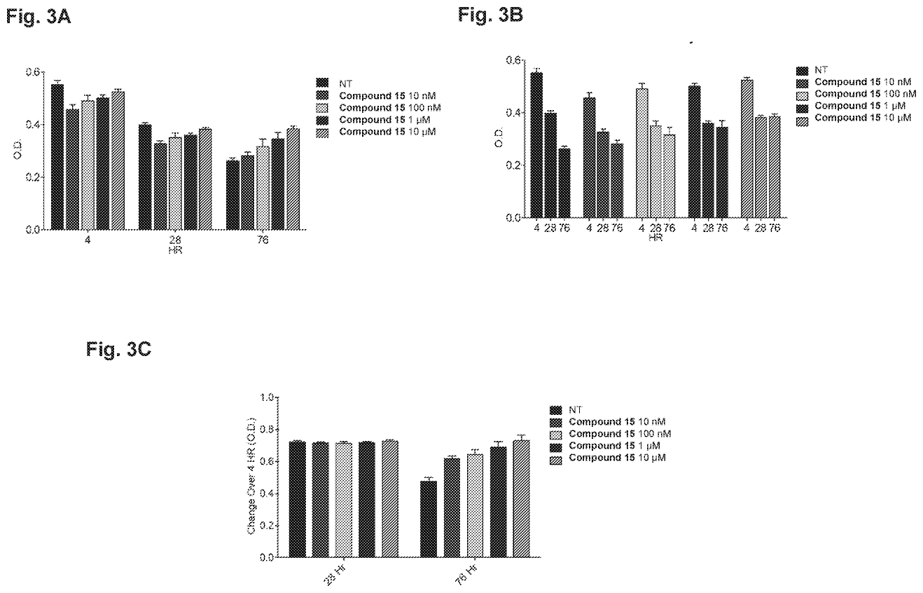

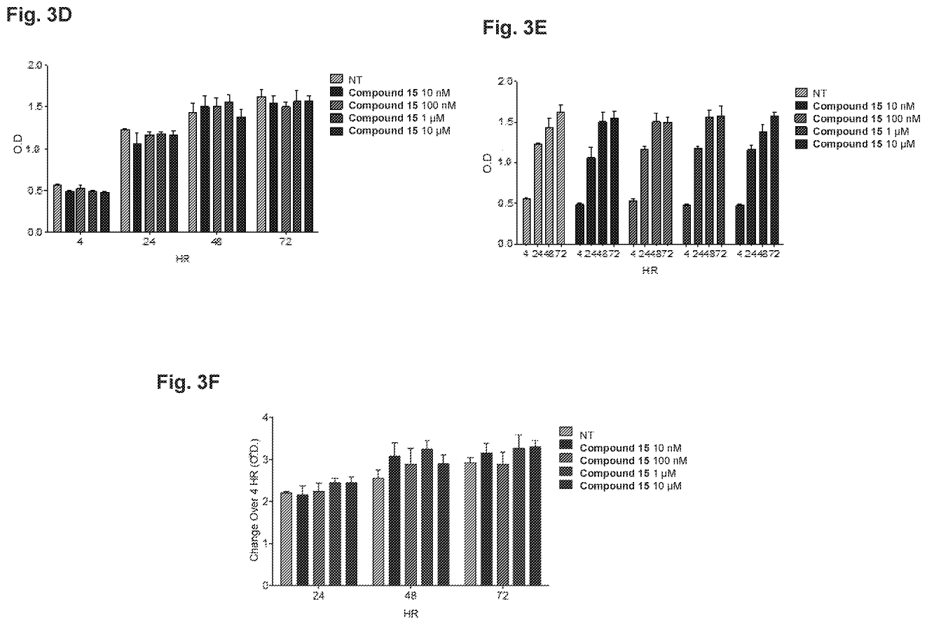

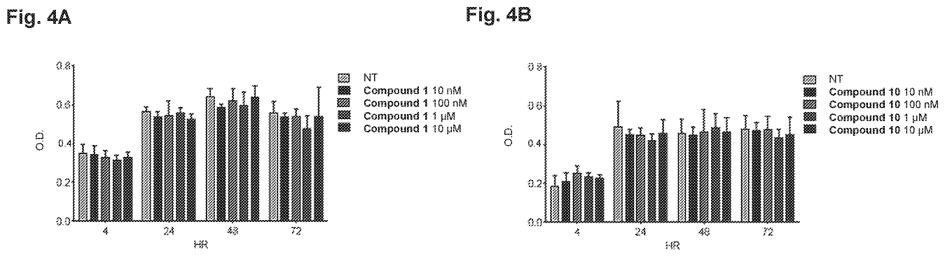

In these assays compound 15 showed no toxicity up to 76 hours in EC (see FIGS. 3A-3C) and up to 72 hours in SMC (see FIGS. 3D-3F). Compounds 1 and 10 also showed no toxicity up to 72 hours in SMC (see FIGS. 4A and 4B respectively).

IFN-γ Gene Expression Induction in NK-92 Cells

IFN-γ gene and protein expression induction is a standard to measure IL-18 activity in NK92 cells (Kalina et al., J. Immunol. 165:1307-1313, 2000).

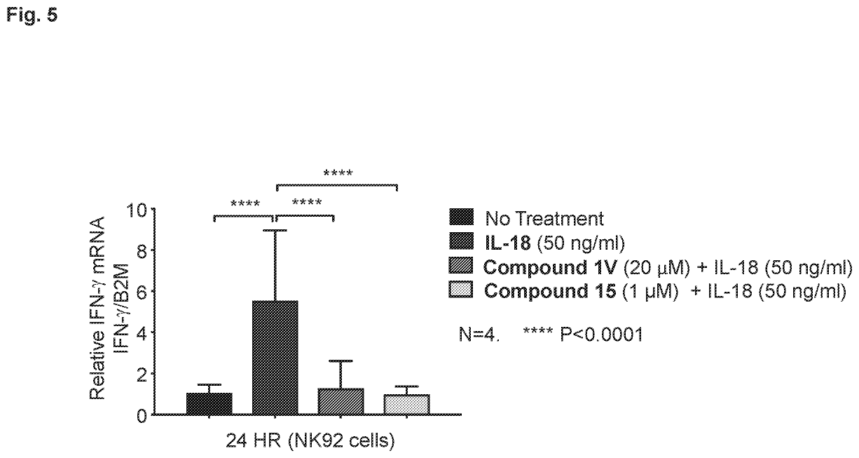

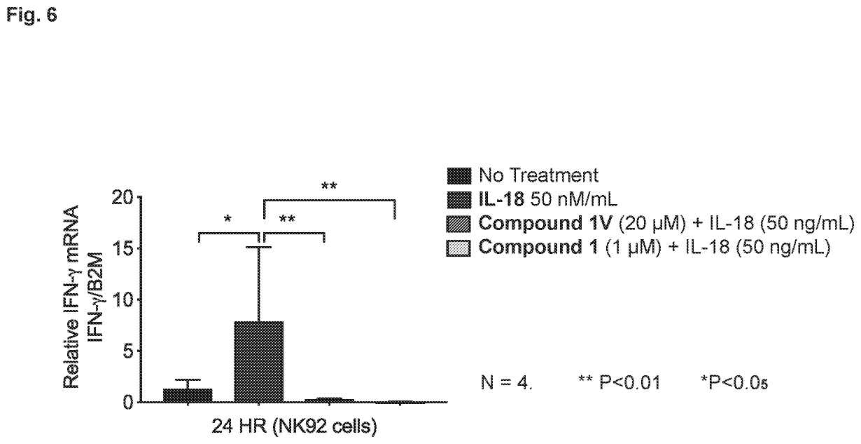

NK92 cells were treated with 1 μM concentration of compounds 1, 10, or 1V. Compound 1V is a known inhibitor of IFN-γ expression in NK92 cells (Kalina et al., J. Immunol. 2000 165, 1307-1313). 24 hours after treatment, total RNA was harvested and qRT-PCR was conducted using a previously published protocols (Raof et al., BMC Genomics, 17-20, 2016; Monahan et al., FASEB J., 23:557-564, 2009) to measure the gene expression of IFN-γ. Comparison of the ligand-induced IFN-γ expression is shown in FIG. 5: IL-18 significantly increases IFN-γ expression (5.52±3.43) vs. no treatment (1.06±0.38); compound 15 (1 μM) significantly decreases IFN-γ expression (0.99±0.38) vs. IL-18 treated cells. FIG. 6 shows IFN-γ expression inhibition results: IL-18 significantly increases IFN-γ expression (7.9±7.23) vs. no treatment (1.33±0.85); compound 1 (1 μM) significantly decreases IFN-γ expression (0.04±0.02) vs. IL-18 treated cells; 1V is a positive control and a known IFN-γ expression inhibitor.

### Microscale Thermophoresis

Recombinant IL18 was fluorescently labelled using the lysine reactive Pico RED NT647 dye from NanoTemper Technologies and stored in a PBS buffer containing 0.05% TWEEN (Buffer A). Stock concentration of labelled IL18 was 10 M. Working concentrations of IL18 were diluted in Buffer A to 10, 40, or 50 nM. Except for the positive control, IL18BP, stock concentration for all compounds, or ligands, was 10-25 mM in 100% DMSO. Ligands were diluted in Buffer A to working concentrations of 50-500 μM. Containing 0.8-2% DMSO. Stock concentration of IL8BP, was 50 μM, stored in 20 mM Tris-HI buffer (pH 8.0) containing 0.4 M Urea and 10% glycerol (Buffer B). Prior to MST experiments being performed 16 preparations of two-fold serial dilutions of ligand in uniform buffer, starting with the working Uigand concentration, were incubated with equal volumes of a fixed concentration of labelled IL8. After 30 minutes incubation at room temperature, 16 Monolith NT. 15 standard capillaries were loaded with 10 μl volumes of each of the ligand/IL18 preparations. Tests described in Results were run at 3700.

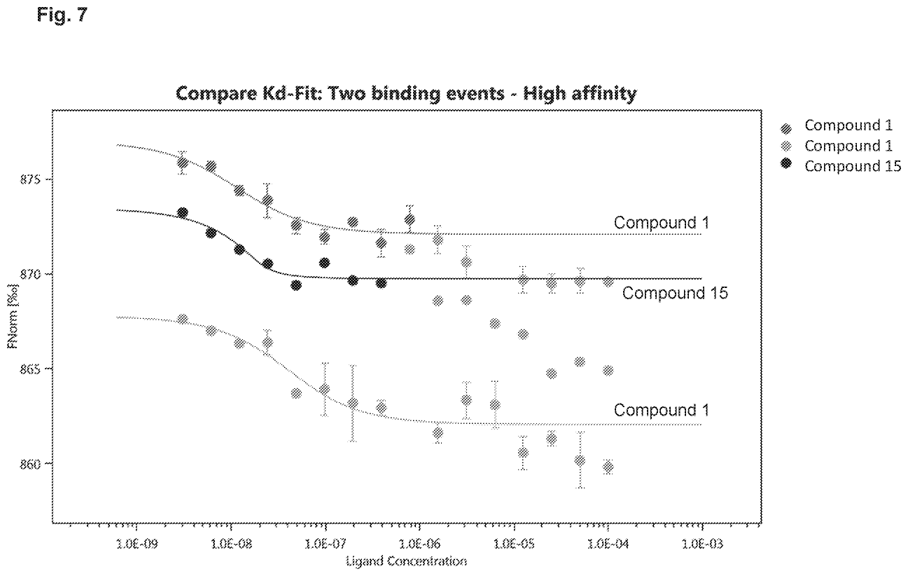

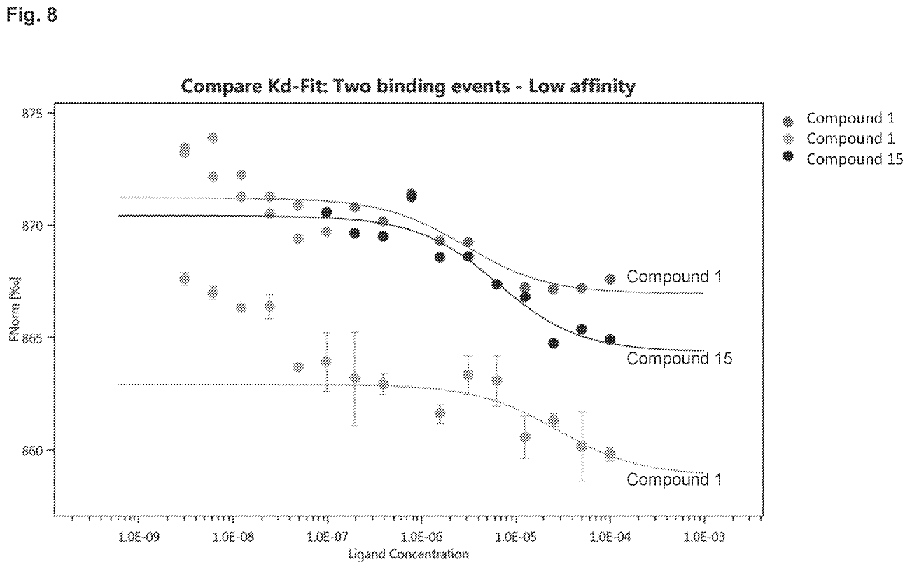

Experiments were performed on a Monolith NT.115Pico machine (NanoTemper Technologies GmbH, Germany). Data quality and analysis and graphic rendering were performed using MO.Affinity Analysis v2.3 software from NanoTemper technologies. Evaluation strategy for all analyses was standardized to measure relative change of fluorescence (FNorm %) over a 2.5 second of laser induced temperature shift per capillary. Statistical analysis was performed using NanoTemper's MOControl software. The results of this assay for compounds 1, 10, and 15 are shown in FIGS. 7, 8, 9A, 9B, and 90 and Table 3 describes the MST assay conditions.

TABLE 3

| | | | | |
|---|---|---|---|---|
| Name: | IL18BP | 1 | 10 | 15 |
| Target Name: | IL18 | IL18 | IL18 | IL18 |
| Target Concentration: | 5 nM | 20 nM | 20 nM | 20 nM |
| Ligand Name: | BP | CP1 | CP10 | CP15 |
| Ligand Concentration | 25 μM to | 100 μM to | 100 μM to | 100 μM to |
| | 0.000763 μM | 0.00305 μuM | 0.00305 μM | 0.00305 μM |
| n: | 2 | 2 | 2 | 2 |
| Comments: | | | | |
| Excitation Power: | 20% | 5% | 5% | 5% |
| MST Power: | 40% | 40% | 40% | 40% |
| Temperature: | 37.0° C. | 37.0° C. | 37.0° C. | 37.0° C. |
| Kd: | 1.8469E−09 | 6.0575E−08 | 2.2711E−09 | 5.6441E−06 |
| Kd Confidence: | ±1.0427E−09 | ±6.8251E−08 | ±4.1613E−09 | ±2.7051E−06 |
| Response Amplitude: | 4.9133364 | 6.2004726 | 5.5261596 | 7.07334911 |
| TargetConc: | 5E−09[Fixed] | 1.3693E−15 | 5E−09[Fixed] | 2E−08[Fixed] |
| Unbound: | 871.43 | 867.62 | 881.12 | 872.28 |
| Bound: | 866.52 | 861.42 | 875.59 | 865.21 |
| Std. Error of Regression: | 0.52447759 | 1.0917105 | 1.3511843 | 0.98154729 |
| Reduced X²: | 20.871973 | 13.716328 | 10.456252 | 144.08809 |
| Signal to Noise: | 10.062908 | 6.4074464 | 4.3932183 | 7.7408351 |

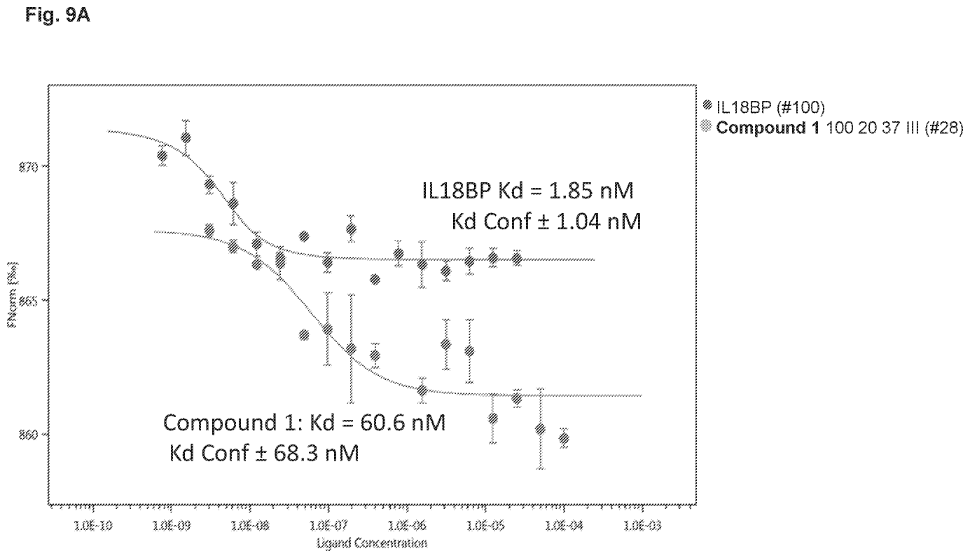

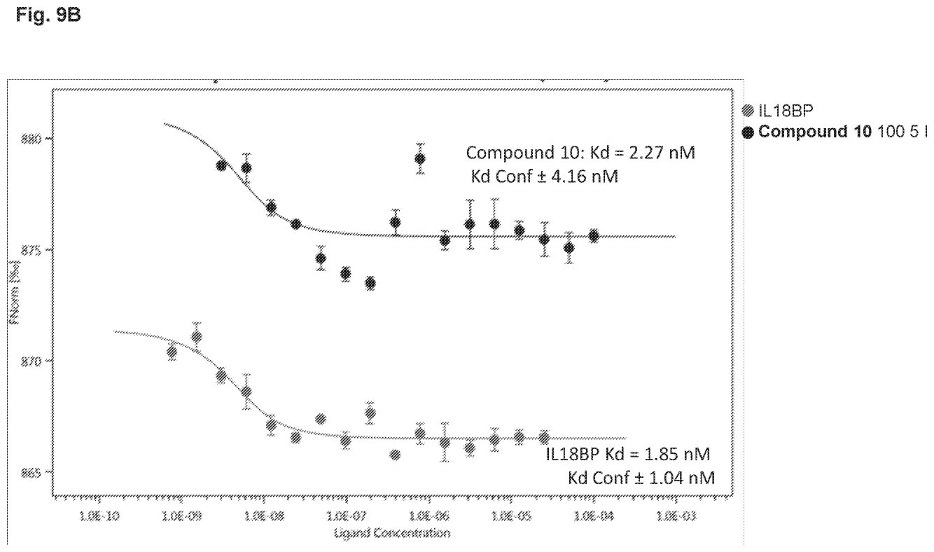

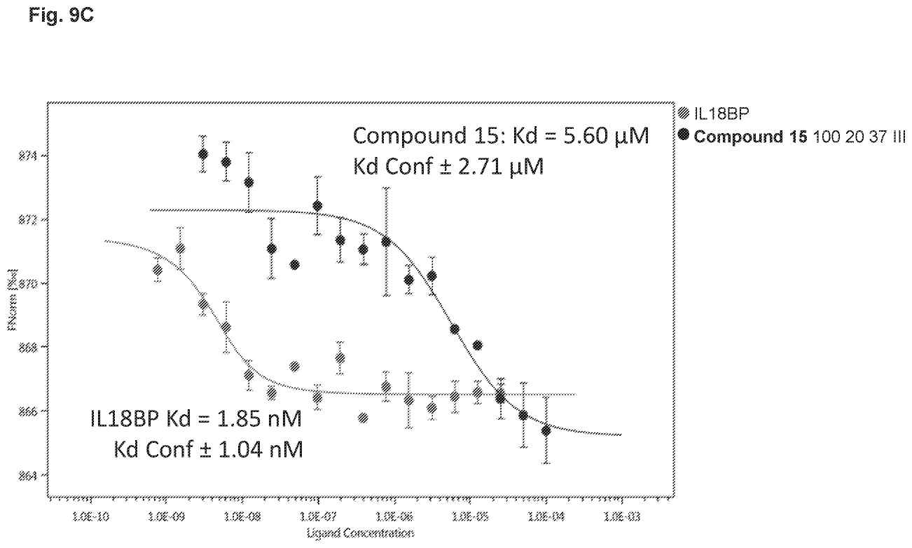

FIG. 9A is graph showing binding affinity events of BP (Kd=1.85 nM; Kd Conf±1.04 nM) and compound 1 (Kd=60.6 nM; Kd Conf±68.3 nM). FIG. 9B is graph illustrating binding affinity events of BP (Kd=1.85 nM; Kd Conf±1.04 nM) and compound 10 (Kd=2.27 nM; Kd Conf±4.16 nM). FIG. 90 is graph illustrating binding affinity events of BP (Kd=1.85 nM; Kd Conf±1.04 nM) and compound 15 (Kd=5.60 μM; Kd Conf±2.71 μM). For all three compounds high affinity binding events occur at low ligand concentration, and low affinity binding events also appear to occur at higher ligand concentrations.

### Example 2. Cytokine Expression in Human Saphenous Vein Ex Vivo

In this example, a cytokine storm/inflammation common to many vascular conditions has been modeled by challenging human saphenous vein segments with lipopolysaccharide (LPS). Discarded human saphenous vein samples were collected from patients undergoing peripheral bypass grafting.

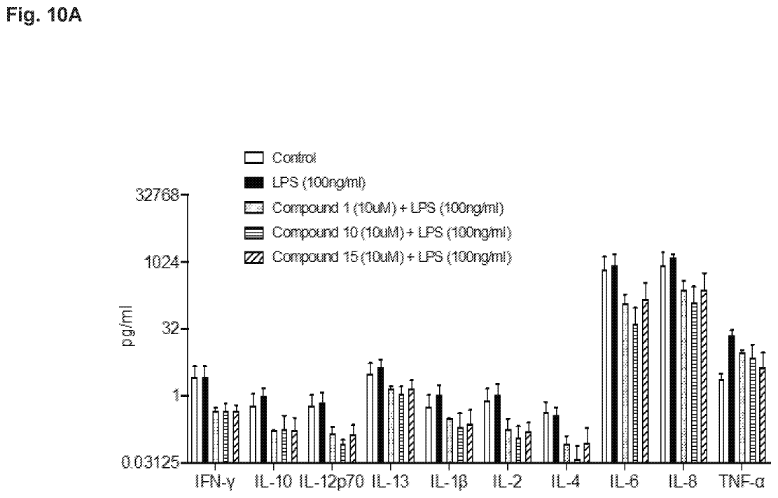

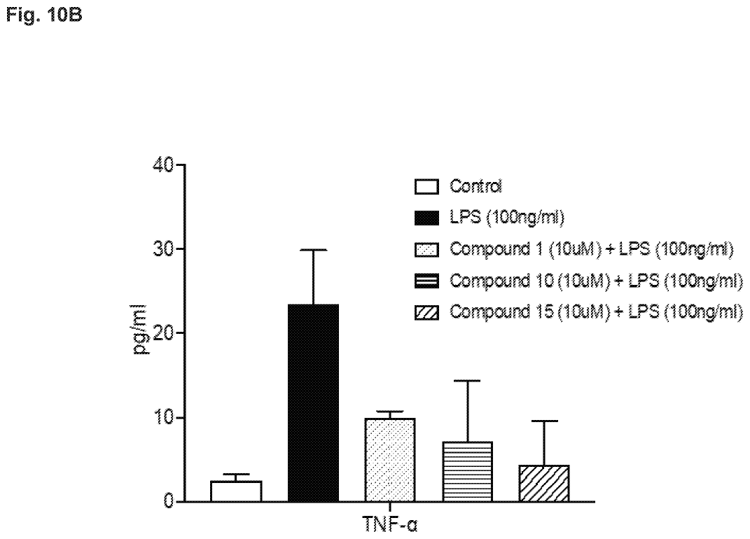

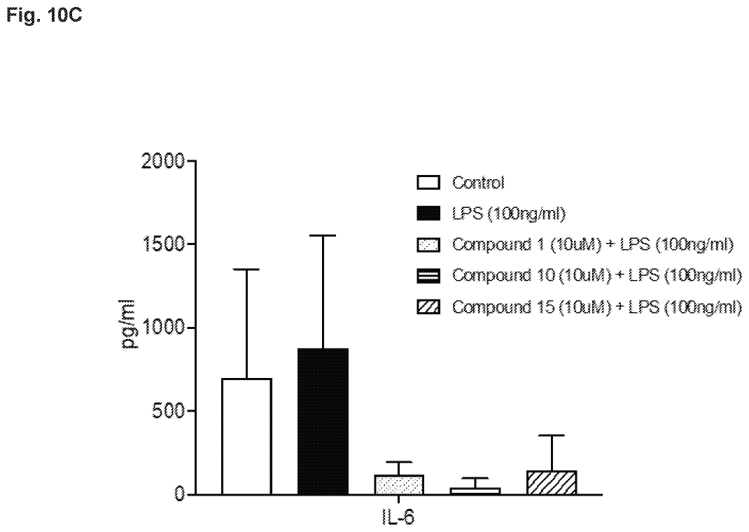

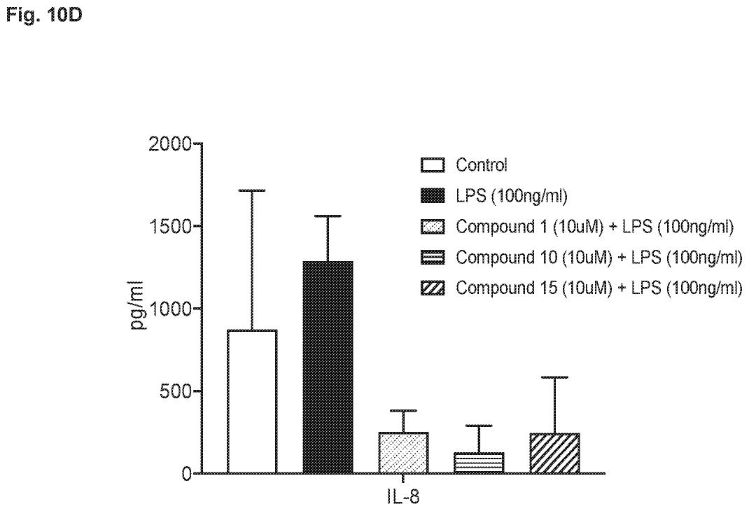

Samples were subjected to Human (h) TheRPA (Therapeutic Responses Predictor Assay) which is based on ex-vivo treatment of biopsies (explant cultures). The tissues were transferred to the lab within 30 min of their harvesting and cultured in DMEM+10% FBS+1% antibiotic/antimycotic (DMEM plus glucose, glutamine, sodium pyruvate: Gibco 11995-065; Heat Inactivated FBS: Gibco 16140-089; Antibiotic-Antimycotic: Gibco 15240-096) in the presence of vehicle, LPS (100 ng/ml) or LPS (100 ng/ml)+compound 1/compound 10/compound 15 (10 μM). After 18 hours, culture supernatants were collected and kept at −80° C. Cytokine levels in cell culture supernatant were measured by multiplex ELISA (Milliplex map human cytokine/chemokine magnetic bead kit from Millipore). The results of this test are shown in FIGS. 10A-10D. Compounds 1, 5, and 10 all significantly inhibited IL-6, IL-8 and TNF expression. Additionally, all three compounds also inhibited the endogenous expression of other cytokines including IFN-γ, IL-13, and IL-2.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following enumerated paragraphs:

1. A compound of formula (I):

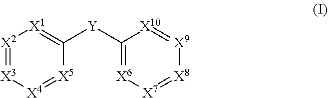

or a pharmaceutically acceptable salt thereof,
wherein:

each of X¹, X², X³, X⁴, X⁵, X⁶, X⁷, X⁸, X⁹, and X¹⁰ is independently CR¹ or N;

Y is O, C(R²)₂, or S(O)ₜ;

each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R² is independently hydrogen or optionally substituted C₁-C₆ alkyl; or both R² combine to form ═O, ═S, or ═NR⁵;

R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each R⁴ is independently optionally substituted C₁-C₆ alkyl, optionally substituted C₆-C₁₀ aryl, optionally substituted heteroaryl, or optionally substituted heterocyclyl;

R⁵ is hydrogen, hydroxyl, —S(O)₂R⁴, or optionally substituted C₁-C₆ alkyl;

each t is independently 0, 1, or 2; and

the compound of formula (I) is not a compound selected from the group consisting of:

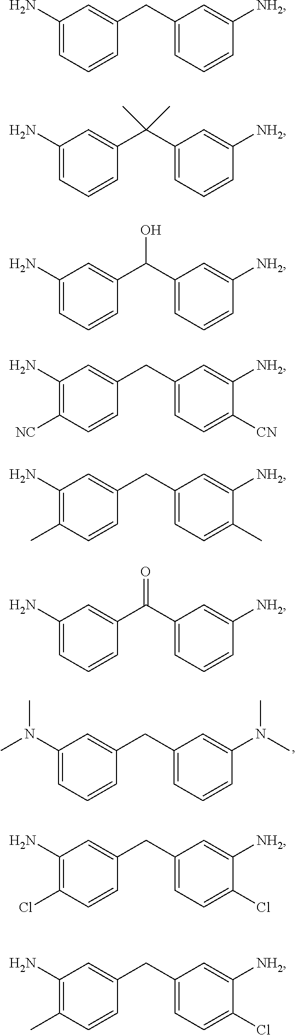

and pharmaceutically acceptable salts thereof; and
provided that at least one of X¹, X², X³, X⁴, and X⁵ is CR¹, at least one of X⁶, X⁷, X⁸, X⁹, and X¹⁰ is CR¹, and at least one R¹ is halogen, hydroxyl, cyano, nitro, —N(R³)₂, —S(O)ₜR⁴, —OR³, or optionally substituted C₁-C₆ alkyl.
2. The compound of paragraph 1, wherein Y is C(R²)₂.
3. The compound of paragraph 1 or 2, wherein at least one R² is hydrogen.
4. The compound of paragraph 1 or 2, wherein both R² combine to form ═O.
5. The compound of paragraph 1 or 2, wherein each R² is independently optionally substituted C₁-C₆alkyl.
6. The compound of any one of paragraphs 1 to 5, wherein each of X² and X⁹ is independently CR¹.
7. The compound of any one of paragraphs 1 to 6, wherein at least two R¹ groups are —N(R³)₂.
8. The compound of paragraph 1, wherein the compound is a compound of formula (Ia):

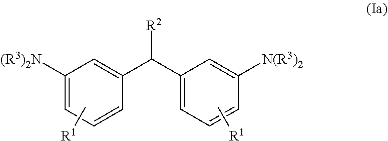

or a pharmaceutically acceptable salt thereof,
wherein:

each R¹ is independently halogen, hydroxyl, cyano, nitro, —N(R³)₂, —S(O)ₜR⁴, —OR³, optionally substituted C₁-C₆ alkyl, optionally substituted C₆-C₁₀aryl, optionally substituted C₃-C₈cycloalkyl, or optionally substituted heteroaryl.

9. The compound of paragraph 1, wherein the compound is a compound of formula (Ib):

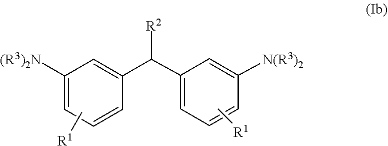

or a pharmaceutically acceptable salt thereof.
10. The compound of paragraph 8 or 9, wherein R² is hydrogen, hydroxyl, or optionally substituted C₁-C₆ alkyl.
11. A pharmaceutical composition comprising a compound of formula (I):

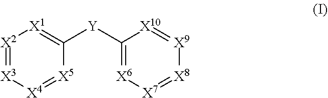

or a pharmaceutically acceptable salt thereof,
wherein:
each of X¹, X², X³, X⁴, X⁵, X⁶, X⁷, X⁸, X⁹, and X¹⁰ is independently CR¹ or N;
Y is O, C(R²)₂, or S(O)ₜ;
each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each R² is independently hydrogen, hydroxyl, or optionally substituted C₁-C₆alkyl; or both R² combine to form ═O, ═S, or ═NR⁵;
R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each R⁴ is independently optionally substituted C₁-C₆ alkyl, optionally substituted C₆-C₁₀ aryl, optionally substituted heteroaryl, or optionally substituted heterocyclyl;
R⁵ is hydrogen, hydroxyl, —S(O)₂R⁴, or optionally substituted C₁-C₆ alkyl; and
each t is independently 0, 1, or 2.
12. The pharmaceutical composition of paragraph 11, wherein Y is C(R²)₂.
13. The pharmaceutical composition of paragraph 11 or 12, wherein at least one R² is hydrogen.
14. The pharmaceutical composition of paragraph 11 or 12, wherein both R² combine to form ═O.
15. The pharmaceutical composition of paragraph 11 or 12, wherein each R² is independently optionally substituted C₁-C₆ alkyl.
16. The pharmaceutical composition of any one of paragraphs 11 to 15, wherein each of X² and X⁹ is independently CR¹.
17. The pharmaceutical composition of any one of paragraphs 11 to 16, wherein at least two R¹ groups are —N(R³)₂.
18. The pharmaceutical composition of paragraph 11, wherein the compound is a compound of formula (Ia):

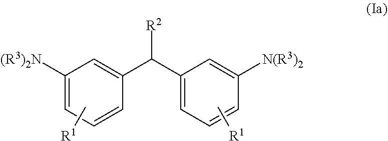

or a pharmaceutically acceptable salt thereof,
wherein:

each R¹ is independently halogen, hydroxyl, cyano, nitro, —N(R³)₂, —S(O)ₜR⁴, —OR³, optionally substituted C₁-C₆ alkyl, optionally substituted C₆-C₁₀aryl, optionally substituted C₃-C₈cycloalkyl, or optionally substituted heteroaryl.

19. The pharmaceutical composition of paragraph 11, wherein the compound is a compound of formula (Ib):

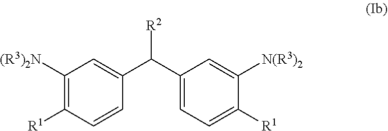

or a pharmaceutically acceptable salt thereof.
20. The pharmaceutical composition of paragraph 18 or 19, wherein R² is hydrogen, hydroxyl, or optionally substituted C₁-C₆ alkyl.
21. A pharmaceutical composition comprising a compound selected from the group consisting of:

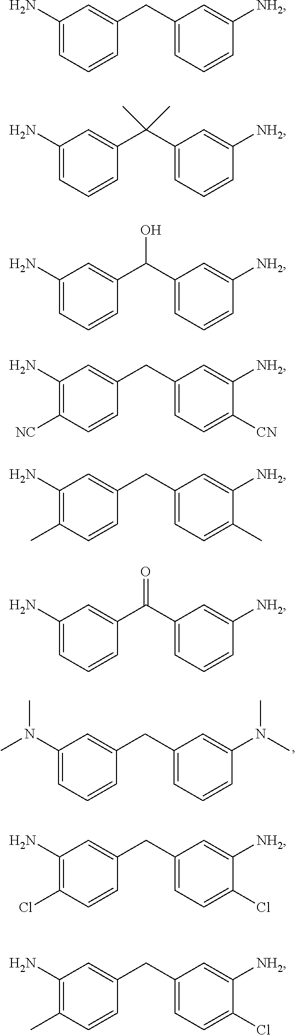

and pharmaceutically acceptable salts thereof.
22. A compound of formula (II):

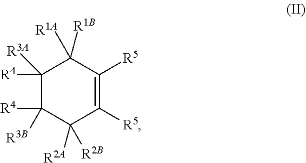

or a pharmaceutically acceptable salt thereof,
wherein:

(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or

(ii) each of R^{1A} and R^{2A} is independently hydrogen, halogen, hydroxyl, cyano, nitro, —S(O)ₜR⁶, or —OR⁷; R^{1B} and R^{3A}, together with the atoms to which they are attached, combine to form a double bond; and R^{2B} and R^{3B}, together with the atoms to which they are attached, combine to form a double bond;

both R⁴, together with the atoms to which they are attached, combine to form an optionally substituted heterocyclic ring;

both R⁵, together with the atoms to which they are attached, combine to form an optionally substituted heterocyclic ring;

each R⁶ is independently hydrogen, halogen, cyano, or optionally substituted C₁-C₆ alkyl;

each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;

each t is independently 0, 1, or 2; and

the compound of formula (II) is not a compound selected from the group consisting of:

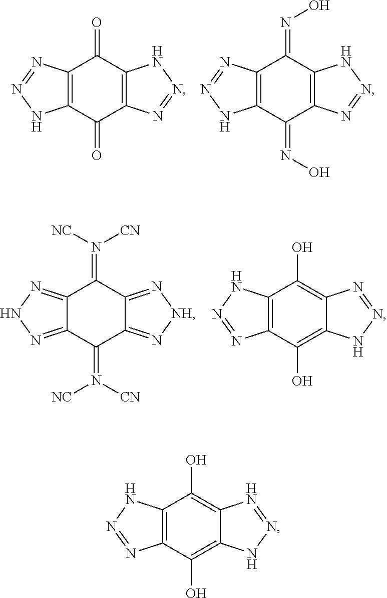

pharmaceutically acceptable salts thereof.
23. The compound of paragraph 22, wherein both R⁴, together with the atoms to which they are attached, combine to form an optionally substituted heterocyclic ring comprising an endocyclic nitrogen atom.
24. The compound of paragraph 22 or 23, wherein both R⁴, together with the atoms to which they are attached, combine to form an optionally substituted, 5-membered heterocyclic ring.
25. The compound of any one of paragraphs 22 to 24, wherein both R⁵, together with the atoms to which they are attached, combine to form an optionally substituted heterocyclic ring comprising an endocyclic nitrogen atom.
26. The compound of any one of paragraphs 22 to 25, wherein both R⁵, together with the atoms to which they are attached, combine to form an optionally substituted, 5-membered heterocyclic ring.
27. The compound of paragraph 22, wherein the compound is a compound of formula (IIa):

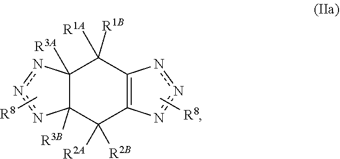

or a pharmaceutically acceptable salt thereof, and each R⁸ is independently H or optionally substituted C₁-C₆ alkyl.
28. The compound of any one of paragraphs 22 to 27, wherein R^{1A} and R^{1B} combine to form ═O.
29. The compound of any one of paragraphs 22 to 28, wherein R^{2A} and R^{2B} combine to form ═O.
30. The compound of any one of paragraphs 22 to 27, wherein each of R^{1A} and R^{2A} is independently hydrogen, halogen, or hydroxyl.
31. A pharmaceutical composition comprising a compound of formula (II):

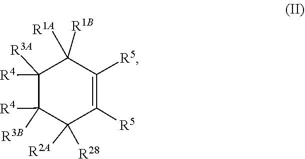

or a pharmaceutically acceptable salt thereof,
wherein:

(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or

(ii) each of R^{1A} and R^{2A} is independently hydrogen, halogen, hydroxyl, cyano, nitro, —S(O)R⁶, or —OR⁷; R^{1B} and R^{3A}, together with the atoms to which they are attached, combine to form a double bond; and R^{2B} and R^{3B}, together with the atoms to which they are attached, combine to form a double bond;

both R⁴, together with the atoms to which they are attached, combine to form an optionally substituted heterocyclic ring;

both R⁵, together with the atoms to which they are attached, combine to form an optionally substituted heterocyclic ring;

each R⁶ is independently hydrogen, halogen, cyano, or optionally substituted C₁-C₆ alkyl;

each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl; and

each t is independently 0, 1, or 2.

32. The pharmaceutical composition of paragraph 31, wherein both R⁴, together with the atoms to which they are attached, combine to form an optionally substituted heterocyclic ring comprising an endocyclic nitrogen atom.
33. The pharmaceutical composition of paragraph 31 or 32, wherein both R⁴, together with the atoms to which they are attached, combine to form an optionally substituted, 5-membered heterocyclic ring.
34. The pharmaceutical composition of any one of paragraphs 31 to 33, wherein both R⁵, together with the atoms to which they are attached, combine to form an optionally substituted heterocyclic ring comprising an endocyclic nitrogen atom.

35. The pharmaceutical composition of any one of paragraphs 31 to 34, wherein both R⁵, together with the atoms to which they are attached, combine to form an optionally substituted, 5-membered heterocyclic ring.

36. The pharmaceutical composition of paragraph 31, wherein the compound is a compound of formula (IIa):

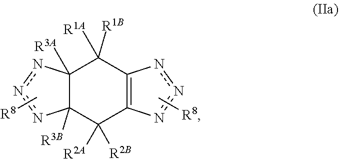

or a pharmaceutically acceptable salt thereof, and each R⁸ is independently H or optionally substituted C₁-C₆ alkyl.
37. The pharmaceutical composition of any one of paragraphs 31 to 36, wherein R^{1A} and R^{1B} combine to form ═O.
38. The pharmaceutical composition of any one of paragraphs 31 to 37, wherein R^{2A} and R^{2B} combine to form ═O.
39. The pharmaceutical composition of any one of paragraphs 31 to 36, wherein each of R^{1A} and R^{2A} is independently hydrogen, halogen, or hydroxyl.
40. A pharmaceutical composition comprising a compound selected from the group consisting of:

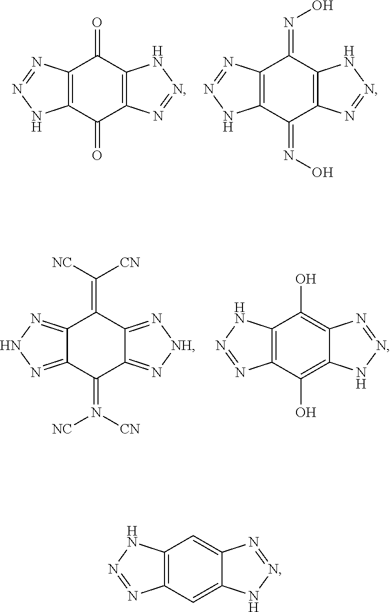

and pharmaceutically acceptable salts thereof.
41. A compound of formula (III):

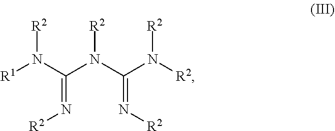

or a pharmaceutically acceptable salt thereof,
wherein:

R¹ is an optionally substituted C₆-C₁₀ aryl or optionally substituted heteroaryl;

each R² is independently hydrogen or an optionally substituted C₁-C₆ alkyl; and

the compound of formula (III) is not a compound selected from the group consisting of:

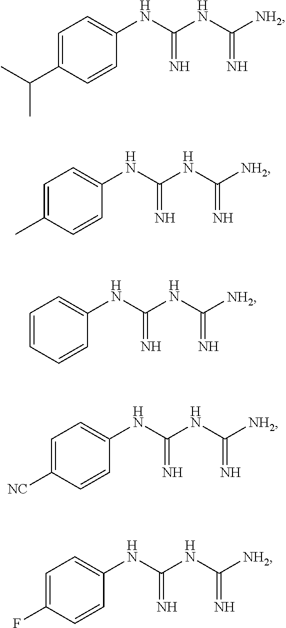 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]

and pharmaceutically acceptable salts thereof.
42. The compound of paragraph 41, wherein R¹ is optionally substituted C₆-C₁₀ aryl.
43. The compound of paragraph 41 or 42, wherein each R² is hydrogen.
44. The compound of paragraph 41, wherein the compound is a compound of formula (IIIa):

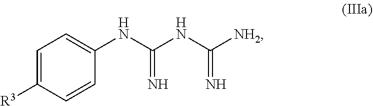

or a pharmaceutically acceptable salt thereof,
wherein:
R³ is optionally substituted C₁-C₆ alkyl, halogen, —OR⁴, —S(O)R⁵, or —N(R⁴)₂;
each R⁴ is independently hydrogen or optionally substituted C₁-C₆ alkyl;

R⁵ is optionally substituted C₁-C₆ alkyl, optionally substituted C₆-C₁₀ aryl, or optionally substituted heteroaryl.

45. The compound of paragraph 44, wherein R³ is optionally substituted C₁-C₆ alkyl or halogen.
46. A pharmaceutical composition comprising a compound of formula (III):

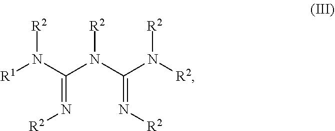

or a pharmaceutically acceptable salt thereof,
wherein:

R¹ is an optionally substituted C₆-C₁₀ aryl or optionally substituted heteroaryl; and

each R² is independently hydrogen or an optionally substituted C₁-C₆ alkyl.

47. The pharmaceutical composition of paragraph 46, wherein R¹ is optionally substituted C₆-C₁₀ aryl.
48. The pharmaceutical composition of paragraph 46 or 47, wherein each R² is hydrogen.
49. The pharmaceutical composition of paragraph 46, wherein the compound is a compound of formula (IIIa):

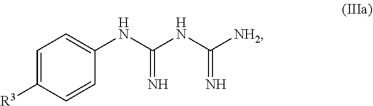

or a pharmaceutically acceptable salt thereof,
wherein:

R³ is hydrogen, optionally substituted C₁-C₆ alkyl, halogen, cyano, —OR⁴, —S(O)R⁵, or —N(R⁴)₂;

each R⁴ is independently hydrogen or optionally substituted C₁-C₆ alkyl;

R⁵ is optionally substituted C₁-C₆ alkyl, optionally substituted C₆-C₁₀ aryl, or optionally substituted heteroaryl; and

each t is independently 0, 1, or 2.

50. The pharmaceutical composition of paragraph 49, wherein R³ is optionally substituted C₁-C₆ alkyl, hydrogen, halogen, or cyano.
51. A pharmaceutical composition comprising a compound selected from the group consisting of:

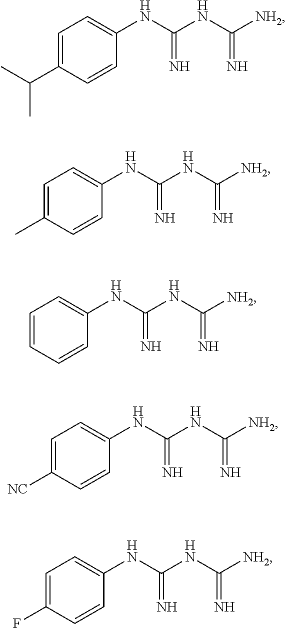 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]

and pharmaceutically acceptable salts thereof.

52. A method of inhibiting interaction between IL18 and IL18R in a medium comprising IL18 and a cell expressing IL18R, the method comprising contacting the cell in the medium with a compound of any one of paragraphs 1 to 51, wherein after the contacting step the interaction between IL18 and IL18R is inhibited.

53. The method of paragraph 52, wherein the cell is in a subject.
54. The method of paragraph 52 or 53, wherein the medium is a tissue or bodily fluid of a subject.
55. A method of treating an inflammatory or autoimmune disorder in a subject in need thereof, the method comprising administering a therapeutically effective amount of the compound of any one of paragraphs 1 to 10, 22-30, and 41-45, or a pharmaceutically acceptable salt thereof, or the pharmaceutical composition of any one of paragraphs 11 to 21, 31 to 40, and 46-51, to the subject.
56. The method of paragraph 55, wherein the inflammatory or autoimmune disorder is selected from the group consisting of rheumatoid arthritis, osteoarthritis, lupus, psoriasis, inflammatory bowel disease, vascular graft failure, heart disease, vascular disease, type 1 diabetes, type 2 diabetes, metabolic syndrome, diabetic wound healing, trauma, burn wounds, Steven Johnsons syndrome, and kidney disease.
57. The method of paragraph 56, wherein the inflammatory or autoimmune disorder is a vascular graft failure.
58. The method of paragraph 57, wherein the vascular graft failure is a peripheral vascular graft failure.
59. The method of paragraph 58, wherein the peripheral vascular graft failure is a vein graft failure or prosthetic graft failure.
60. The method of paragraph 57, wherein the vascular graft failure is a coronary artery graft failure.
61. The method of paragraph 60, wherein the coronary graft failure is an is artery graft failure, vein graft failure, prosthetic graft failure.
62. The method of paragraph 57, wherein the vascular graft failure is restenosis after stent graft.
63. The method of paragraph 57, wherein the vascular graft failure is restenosis after balloon angioplasty.
64. The method of paragraph 56, wherein the inflammatory or autoimmune disorder is a heart disease.
65. The method of paragraph 64, wherein the heart disease is cardiomyopathy, congestive heart failure, or ischemic coronary heart disease.
66. The method of paragraph 55, wherein the inflammatory or autoimmune disorder is a vascular disease.
67. The method of paragraph 66, wherein the vascular disease is intimal hyperplasia, atherosclerosis, coronary artery disease, restenosis, primary hypertension, secondary hypertension, peripheral vascular disease, or critical limb-threatening ischemia.
68. The method of paragraph 56, wherein the inflammatory or autoimmune disorder is type 1 diabetes, type 2 diabetes, metabolic syndrome, or diabetic wound healing.
69. The method of paragraph 56, wherein the inflammatory or autoimmune disorder is a kidney disease.
70. The method of paragraph 69, wherein the kidney disease is a chronic kidney disease, end-stage renal disease, or kidney failure.
71. The method of paragraph 55, wherein the inflammatory or autoimmune disorder is a cytokine storm.
72. The method of paragraph 55, wherein the inflammatory or autoimmune disorder is endothelialitis.
73. The method of paragraph 55, 71, or 72, wherein the subject is suffering from acute respiratory distress syndrome (ARDS).
74. The method of paragraph 55, 71, 72, or 73, wherein the inflammatory or autoimmune disorder is associated with an infection.
75. The method of paragraph 74, wherein the infection is a beta-coronavirus infection.
76. The method of paragraph 75, wherein the infection is SARS-CoV-2, SARS-CoV, or MERS-CoV.
77. The method of paragraph 76, wherein the infection is SARS-CoV-2.
78. The method of paragraph 55, wherein the inflammatory or autoimmune disorder is sepsis.
79. The method of paragraph 55, 71, or 78, wherein the inflammatory or autoimmune disorder is associated with a viremia, bacteremia, protozoan infection, or fungal infection.
80. A method of treating a cancer in a subject in need thereof, the method comprising administering a therapeutically effective amount of the compound of any one of paragraphs 1 to 10, 22-30, and 41-45, or a pharmaceutically acceptable salt thereof, or the pharmaceutical composition of any one of paragraphs 11 to 21, 31 to 40, and 46-51, to the subject.
81. The method of paragraph 80, wherein the cancer is gastric cancer, colon cancer, melanoma, and multiple myeloma.

Other embodiments are within the scope of the claims.

What is claimed is:

1. A compound of formula (I): 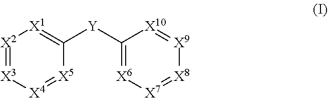
or a pharmaceutically acceptable salt thereof,
wherein:
each of X¹, X², X³, X⁴, X⁵, X⁶, X⁷, X⁸, X⁹, and X¹⁰ is independently CR¹ or N;
Y is O, C(R²)₂, or S(O)ₜ;
each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each R² is independently hydrogen or optionally substituted C₁-C₆ alkyl; or both R² combine to form ═O, ═S, or ═NR⁵;
R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each R⁴ is independently optionally substituted C₁-C₆ alkyl, optionally substituted C₆-C₁₀ aryl, optionally substituted heteroaryl, or optionally substituted heterocyclyl;
R⁵ is hydrogen, hydroxyl, —S(O)₂R⁴, or optionally substituted C₁-C₆ alkyl;
each t is independently 0, 1, or 2; and
the compound of formula (I) is not a compound selected from the group consisting of: 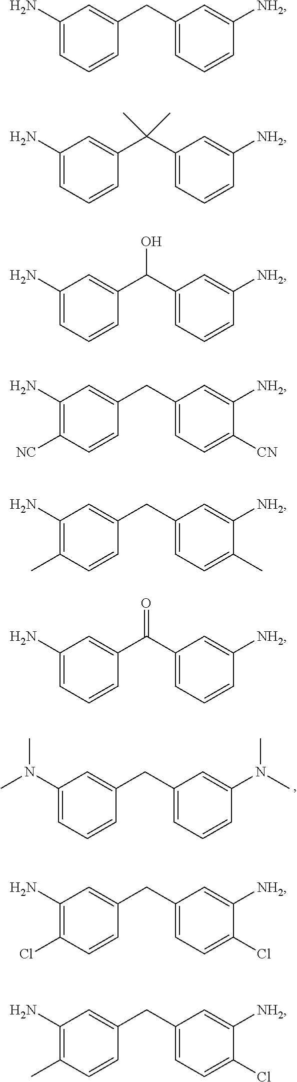
and pharmaceutically acceptable salts thereof; and
provided that at least one of X¹, X², X³, X⁴, and X⁵ is CR¹, at least one of X⁶, X⁷, X⁸, X⁹, and X¹⁰ is CR¹, and at least one R¹ is halogen, hydroxyl, cyano, nitro, —N(R³)₂, —S(O)ₜR⁴, —OR³, or optionally substituted C₁-C₆ alkyl.

2. The compound of claim 1, wherein Y is C(R²)₂.

3. The compound of claim 1, wherein at least one R² is hydrogen.

4. The compound of claim 1, wherein both R² combine to form ═O.

5. The compound of claim 1, wherein each R² is independently optionally substituted C₁-C₆ alkyl.

6. The compound of claim 1, wherein each of X² and X⁹ is independently CR¹.

7. The compound of claim 1, wherein at least two R¹ groups are —N(R³)₂.

8. The compound of claim 1, wherein the compound is a compound of formula (Ia): 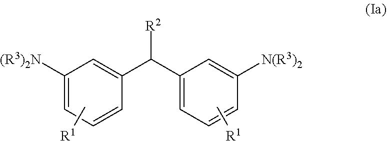
or a pharmaceutically acceptable salt thereof,
wherein:
each R¹ is independently halogen, hydroxyl, cyano, nitro, —N(R³)₂, —S(O)R⁴, —OR³, optionally substituted C₁-C₆ alkyl, optionally substituted C₆-C₁₀aryl, optionally substituted C₃-C₈cycloalkyl, or optionally substituted heteroaryl.

9. The compound of claim 1, wherein the compound is a compound of formula (Ib): 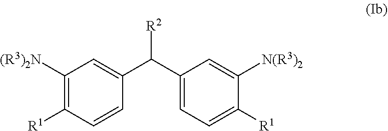
or a pharmaceutically acceptable salt thereof.

10. The compound of claim 8, wherein R² is hydrogen, hydroxyl, or optionally substituted C₁-C₆ alkyl.

11. A pharmaceutical composition comprising a compound of formula (I): 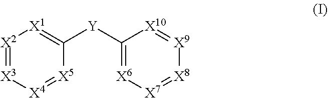
or a pharmaceutically acceptable salt thereof,
wherein:
each of X¹, X², X³, X⁴, X⁵, X⁶, X⁷, X⁸, X⁹, and X¹⁰ is independently CR¹ or N;
Y is O, C(R²)₂, or S(O)ₜ;
each R¹ is independently hydrogen, halogen, hydroxyl, cyano, nitro, —N(R³)₂, —S(O)ₜR⁴, —OR³, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each R² is independently hydrogen, hydroxyl, or optionally substituted C₁-C₆alkyl; or both R² combine to form ═O, ═S, or ═NR⁵;
R³ is hydrogen, —SO₂R⁴, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each R⁴ is independently optionally substituted C₁-C₆ alkyl, optionally substituted C₆-C₁₀ aryl, optionally substituted heteroaryl, or optionally substituted heterocyclyl;
R⁵ is hydrogen, hydroxyl, —S(O)₂R⁴, or optionally substituted C₁-C₆ alkyl; and
each t is independently 0, 1, or 2.

12. The pharmaceutical composition of claim 11, wherein Y is C(R²)₂.

13. The pharmaceutical composition of claim 11, wherein at least one R² is hydrogen.

14. The pharmaceutical composition of claim 11, wherein both R² combine to form ═O.

15. The pharmaceutical composition of claim 11, wherein each R² is independently optionally substituted C₁-C₆ alkyl.

16. The pharmaceutical composition of claim 11, wherein each of X² and X⁹ is independently CR¹.

17. The pharmaceutical composition of claim 11, wherein at least two R¹ groups are —N(R³)₂.

18. The pharmaceutical composition of claim 11, wherein the compound is a compound of formula (Ia): 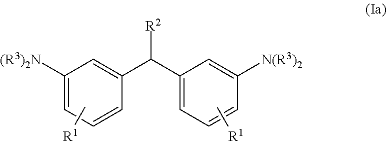
or a pharmaceutically acceptable salt thereof,
wherein:
each R¹ is independently halogen, hydroxyl, cyano, nitro, —N(R³)₂, —S(O)ₜR⁴, —OR³, optionally substituted C₁-C₆ alkyl, optionally substituted C₆-C₁₀aryl, optionally substituted C₃-C₈cycloalkyl, or optionally substituted heteroaryl.

19. The pharmaceutical composition of claim 11, wherein the compound is a compound of formula (Ib): 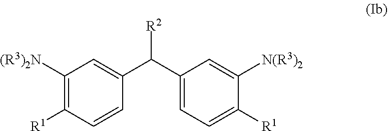
or a pharmaceutically acceptable salt thereof.

20. The pharmaceutical composition of claim 19, wherein R² is hydrogen, hydroxyl, or optionally substituted C₁-C₆ alkyl.

21. A pharmaceutical composition comprising a compound selected from the group consisting of: 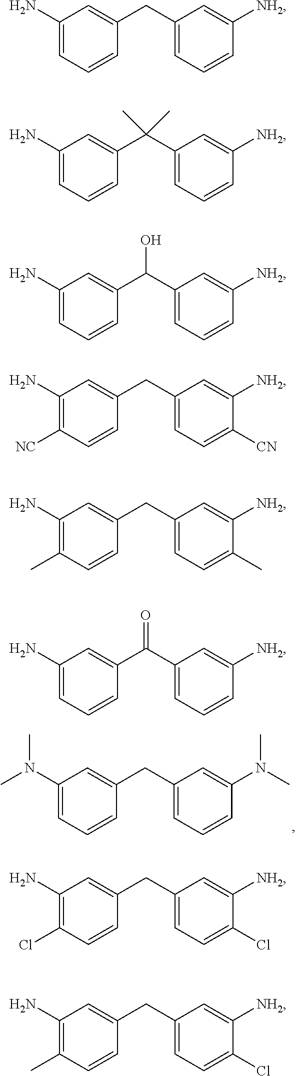
and pharmaceutically acceptable salts thereof.

22. A compound of formula (II): 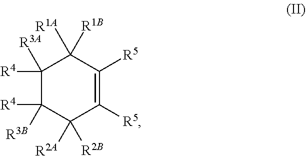
or a pharmaceutically acceptable salt thereof,
wherein:
(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or
(ii) each of R^{1A} and R^{2A} is independently hydrogen, halogen, hydroxyl, cyano, nitro, —S(O)R⁶, or —OR⁷; R^{1B} and R^{3A}, together with the atoms to which they are attached, combine to form a double bond; and R^{2B} and R^{3B}, together with the atoms to which they are attached, combine to form a double bond;
both R⁴, together with the atoms to which they are attached, combine to form an optionally substituted heterocyclic ring;
both R⁵, together with the atoms to which they are attached, combine to form an optionally substituted heterocyclic ring;
each R⁶ is independently hydrogen, halogen, cyano, or optionally substituted C₁-C₆ alkyl;
each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl;
each t is independently 0, 1, or 2; and
the compound of formula (II) is not a compound selected from the group consisting of: 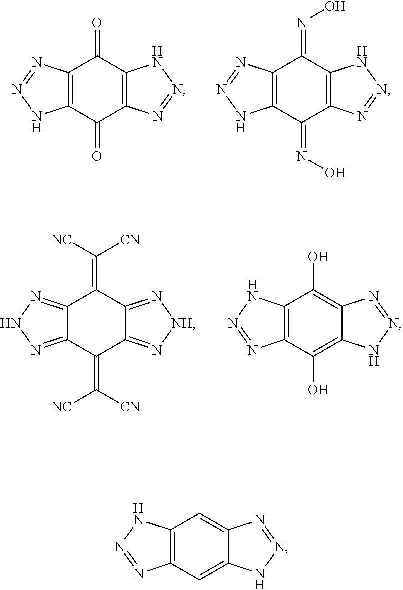
and pharmaceutically acceptable salts thereof.

23. The compound of claim 22, wherein both R⁴, together with the atoms to which they are attached, combine to form an optionally substituted heterocyclic ring comprising an endocyclic nitrogen atom.

24. The compound of claim 22, wherein both R⁴, together with the atoms to which they are attached, combine to form an optionally substituted, 5-membered heterocyclic ring.

25. The compound of claim 22, wherein both R⁵, together with the atoms to which they are attached, combine to form an optionally substituted heterocyclic ring comprising an endocyclic nitrogen atom.

26. The compound of claim 22, wherein both R⁵, together with the atoms to which they are attached, combine to form an optionally substituted, 5-membered heterocyclic ring.

27. The compound of claim 22, wherein the compound is a compound of formula (IIa): 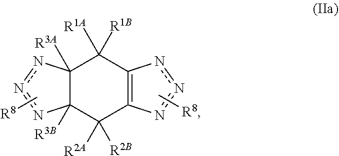
or a pharmaceutically acceptable salt thereof, and each R⁸ is independently H or optionally substituted C₁-C₆ alkyl.

28. The compound of claim 22, wherein R^{1A} and R^{1B} combine to form ═O.

29. The compound of claim 22, wherein R^{2A} and R^{2B} combine to form ═O.

30. The compound of claim 22, wherein each of R^{1A} and R^{2A} is independently hydrogen, halogen, or hydroxyl.

31. A pharmaceutical composition comprising a compound of formula (II): 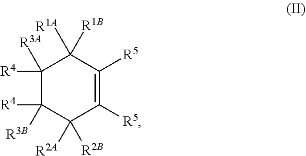
or a pharmaceutically acceptable salt thereof,
wherein:
(i) R^{1A} and R^{1B} combine to form ═O, ═S, ═C(R⁶)₂, or ═NR⁷; R^{2A} and R^{2B} combine to form ═O, ═S, ═C(R⁶)₂, or ═NR⁷; and R^{3A} and R^{3B}, together with the atoms to which they are attached, combine to form a double bond; or
(ii) each of R^{1A} and R^{2A} is independently hydrogen, halogen, hydroxyl, cyano, nitro, —S(O)R⁶, or —OR⁷; R^{1B} and R^{3A}, together with the atoms to which they are attached, combine to form a double bond; and R^{2B} and R^{3B}, together with the atoms to which they are attached, combine to form a double bond;
both R⁴, together with the atoms to which they are attached, combine to form an optionally substituted heterocyclic ring;
both R⁵, together with the atoms to which they are attached, combine to form an optionally substituted heterocyclic ring;
each R⁶ is independently hydrogen, halogen, cyano, or optionally substituted C₁-C₆ alkyl;
each R⁷ is independently hydroxyl, optionally substituted C₁-C₆ alkyl, optionally substituted C₆-C₁₀ aryl, optionally substituted C₃-C₈ cycloalkyl, optionally substituted heteroaryl, or optionally substituted heterocyclyl; and
each t is independently 0, 1, or 2.

32. The pharmaceutical composition of claim 31, wherein both R⁴, together with the atoms to which they are attached, combine to form an optionally substituted heterocyclic ring comprising an endocyclic nitrogen atom.

33. The pharmaceutical composition of claim 31, wherein both R⁴, together with the atoms to which they are attached, combine to form an optionally substituted, 5-membered heterocyclic ring.

34. The pharmaceutical composition of claim 31, wherein both R⁵, together with the atoms to which they are attached, combine to form an optionally substituted heterocyclic ring comprising an endocyclic nitrogen atom.

35. The pharmaceutical composition of claim 31, wherein both R⁵, together with the atoms to which they are attached, combine to form an optionally substituted, 5-membered heterocyclic ring.

36. The pharmaceutical composition of claim 31, wherein the compound is a compound of formula (IIa): 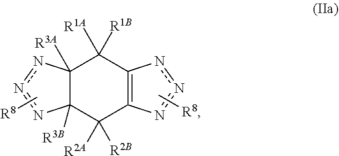
or a pharmaceutically acceptable salt thereof, and each R⁸ is independently H or optionally substituted C₁-C₆ alkyl.

37. The pharmaceutical composition of claim 31, wherein R^{1A} and R^{1B} combine to form ═O.

38. The pharmaceutical composition of claim 31, wherein R^{2A} and R^{2B} combine to form ═O.

39. The pharmaceutical composition of claim 31, wherein each of R^{1A} and R^{2A} is independently hydrogen, halogen, or hydroxyl.

40. A pharmaceutical composition comprising a compound selected from the group consisting of: 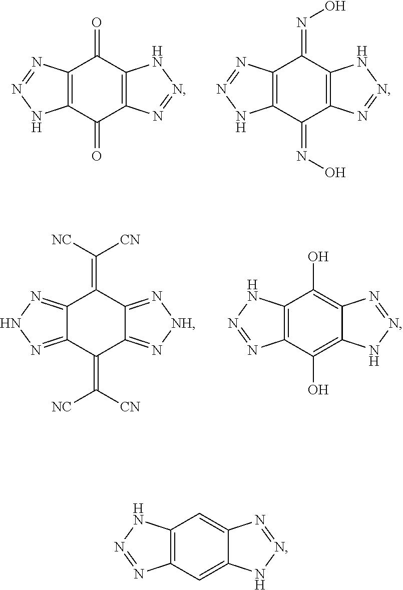
and pharmaceutically acceptable salts thereof.

41. A compound of formula (III): 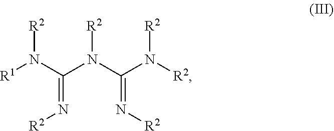
or a pharmaceutically acceptable salt thereof,
wherein:
R¹ is an optionally substituted C₆-C₁₀ aryl or optionally substituted heteroaryl;
each R² is independently hydrogen or an optionally substituted C₁-C₆ alkyl; and
the compound of formula (III) is not a compound selected from the group consisting of: 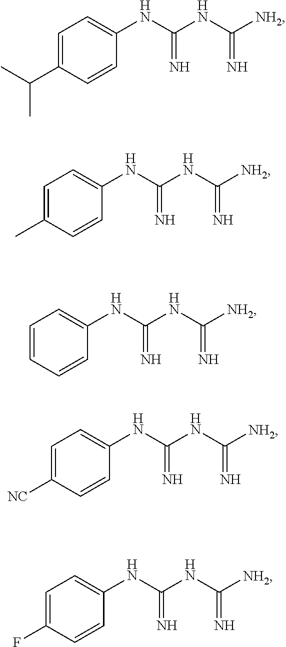 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]
and pharmaceutically acceptable salts thereof.

42. The compound of claim 41, wherein R¹ is optionally substituted C₆-C₁₀ aryl.

43. The compound of claim 41, wherein each R² is hydrogen.

44. The compound of claim 41, wherein the compound is a compound of formula (IIIa): 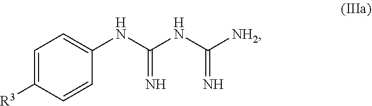
or a pharmaceutically acceptable salt thereof,
wherein:
R³ is optionally substituted C₁-C₆ alkyl, halogen, —OR⁴, —S(O)R⁵, or —N(R⁴)₂;
each R⁴ is independently hydrogen or optionally substituted C₁-C₆ alkyl;
R⁵ is optionally substituted C₁-C₆ alkyl, optionally substituted C₆-C₁₀ aryl, or optionally substituted heteroaryl.

45. The compound of claim 44, wherein R³ is optionally substituted C₁-C₆ alkyl or halogen.

46. A pharmaceutical composition comprising a compound of formula (III): 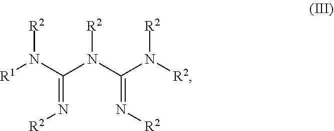
or a pharmaceutically acceptable salt thereof,
wherein:
R¹ is an optionally substituted C₆-C₁₀ aryl or optionally substituted heteroaryl; and
each R² is independently hydrogen or an optionally substituted C₁-C₆ alkyl.

47. The pharmaceutical composition of claim 46, wherein R¹ is optionally substituted C₆-C₁₀ aryl.

48. The pharmaceutical composition of claim 46, wherein each R² is hydrogen.

49. The pharmaceutical composition of claim 46, wherein the compound is a compound of formula (IIIa): 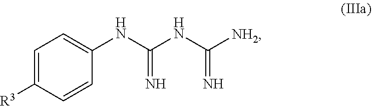
or a pharmaceutically acceptable salt thereof,
wherein:
R³ is hydrogen, optionally substituted C₁-C₆ alkyl, halogen, cyano, —OR⁴, —S(O)R⁵, or —N(R⁴)₂;
each R⁴ is independently hydrogen or optionally substituted C₁-C₆ alkyl;
R⁵ is optionally substituted C₁-C₆ alkyl, optionally substituted C₆-C₁₀ aryl, or optionally substituted heteroaryl; and
each t is independently 0, 1, or 2.

50. The pharmaceutical composition of claim 49, wherein R³ is optionally substituted C₁-C₆ alkyl, hydrogen, halogen, or cyano.

51. A pharmaceutical composition comprising a compound selected from the group consisting of: 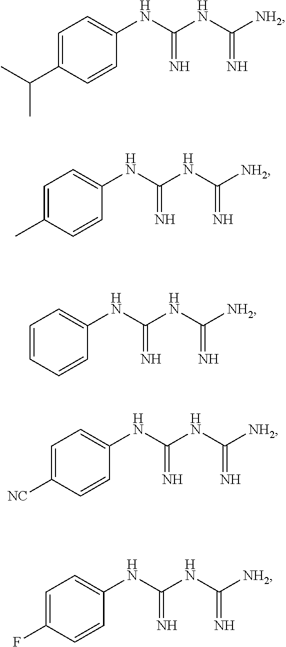 [SMILES: CC(C)c1ccc(NC(=N)NC(=N)N)cc1 ; Cc1ccc(NC(=N)NC(=N)N)cc1 ; N=C(N)NC(=N)Nc1ccccc1 ; N#Cc1ccc(NC(=N)NC(=N)N)cc1 ; N=C(N)NC(=N)Nc1ccc(F)cc1]
and pharmaceutically acceptable salts thereof.

52. A method of inhibiting interaction between IL18 and IL18R in a medium comprising IL18 and a cell expressing IL18R, the method comprising contacting the cell in the medium with a compound of any one of claims 1 to 51, wherein after the contacting step the interaction between IL18 and IL18R is inhibited.

53. The method of claim 52, wherein the cell is in a subject.

54. The method of claim 52, wherein the medium is a tissue or bodily fluid of a subject.

55. A method of treating an inflammatory or autoimmune disorder in a subject in need thereof, the method comprising administering a therapeutically effective amount of the compound of claim 1, or a pharmaceutically acceptable salt thereof, to the subject.

56. The method of claim 55, wherein the inflammatory or autoimmune disorder is selected from the group consisting of rheumatoid arthritis, osteoarthritis, lupus, psoriasis, inflammatory bowel disease, vascular graft failure, heart disease, vascular disease, type 1 diabetes, type 2 diabetes, metabolic syndrome, diabetic wound healing, trauma, burn wounds, Steven Johnsons syndrome, and kidney disease.

57. The method of claim 56, wherein the inflammatory or autoimmune disorder is a vascular graft failure.

58. The method of claim 57, wherein the vascular graft failure is a peripheral vascular graft failure.

59. The method of claim 58, wherein the peripheral vascular graft failure is a vein graft failure or prosthetic graft failure.

60. The method of claim 57, wherein the vascular graft failure is a coronary artery graft failure.

61. The method of claim 60, wherein the coronary graft failure is an is artery graft failure, vein graft failure, prosthetic graft failure.

62. The method of claim 57, wherein the vascular graft failure is restenosis after stent graft.

63. The method of claim 57, wherein the vascular graft failure is restenosis after balloon angioplasty.

64. The method of claim 56, wherein the inflammatory or autoimmune disorder is a heart disease.

65. The method of claim 64, wherein the heart disease is cardiomyopathy, congestive heart failure, or ischemic coronary heart disease.

66. The method of claim 55, wherein the inflammatory or autoimmune disorder is a vascular disease.

67. The method of claim 66, wherein the vascular disease is intimal hyperplasia, atherosclerosis, coronary artery disease, restenosis, primary hypertension, secondary hypertension, peripheral vascular disease, or critical limb-threatening ischemia.

68. The method of claim 56, wherein the inflammatory or autoimmune disorder is type 1 diabetes, type 2 diabetes, metabolic syndrome, or diabetic wound healing.

69. The method of claim 56, wherein the inflammatory or autoimmune disorder is a kidney disease.

70. The method of claim 69, wherein the kidney disease is a chronic kidney disease, end-stage renal disease, or kidney failure.

71. The method of claim 55, wherein the inflammatory or autoimmune disorder is a cytokine storm.

72. The method of claim 55, wherein the inflammatory or autoimmune disorder is endothelialitis.

73. The method of claim 55, wherein the subject is suffering from acute respiratory distress syndrome (ARDS).

74. The method of claim 55, wherein the inflammatory or autoimmune disorder is associated with an infection.

75. The method of claim 74, wherein the infection is a beta-coronavirus infection.

76. The method of claim 75, wherein the infection is SARS-CoV-2, SARS-CoV, or MERS-CoV.

77. The method of claim 76, wherein the infection is SARS-CoV-2.

78. The method of claim 55, wherein the inflammatory or autoimmune disorder is sepsis.

79. The method of claim 55, wherein the inflammatory or autoimmune disorder is associated with a viremia, bacteremia, protozoan infection, or fungal infection.

80. A method of treating a cancer in a subject in need thereof, the method comprising administering a therapeutically effective amount of the compound of claim 1, or a pharmaceutically acceptable salt thereof, to the subject.

81. The method of claim 80, wherein the cancer is gastric cancer, colon cancer, melanoma, and multiple myeloma.
